(19) United States
(12) Patent Application Publication
(10) Pub. No.: US 2020/0246317 A1
(43) Pub. Date: Aug. 6, 2020

(54) METHODS OF TREATING DIABETIC NEUROPATHY WITH A THIAZOLINE ANTI-HYPERALGESIC AGENT

(71) Applicant: CERSCI THERAPEUTICS, INC., Dallas, TX (US)

(72) Inventor: SCOTT L. DAX, LANDENBERG, PA (US)

(21) Appl. No.: 16/654,405

(22) Filed: Oct. 16, 2019

Related U.S. Application Data
(60) Provisional application No. 62/882,372, filed on Aug. 2, 2019.
(60) Provisional application No. 62/800,232, filed on Feb. 1, 2019.

(51) Int. Cl. A61K 31/426; A61K 9/00; A61P 25/02

(52) U.S. Cl. CPC A61K 31/426; A61P 25/02; A61K 9/0053

(57) ABSTRACT

Methods of treating diabetic neuropathy and pain associated with diabetes are provided. The methods include administering to an individual a therapeutically effective amount of a compound of Formula I (Compound 1). The method can be used to treat diabetic neuropathy pain arising from type I or type II diabetes. Compound 1 can be formulated into many suitable dosage forms, including oral dosage forms such as tablets.

### CROSS-REFERENCE TO RELATED APPLICATIONS

The present application claims priority under 35 U.S.C. § 119 (e) to U.S. Provisional Patent Application No. 62/882,372, filed Aug. 2, 2019, and U.S. Provisional Patent Application No. 62/800,232, filed Feb. 1, 2019, which applications are hereby incorporated herein by reference in their entireties.

### BACKGROUND

Pain is defined as an unpleasant sensory and emotional experience. Pain, however, can be informative and useful. For example, nociceptive pain is often indicative of injury (e.g., tissue damage), and such pain typically evokes escape or protective behaviors in animals or in a human, in order to remove itself, or protect itself, from further exposure to the insult. However, inflammation, cellular and neuronal damage and other processes resulting from injury or disease can lead to states of chronic pathological pain. Hyperalgesia is a condition in which enhanced sensitivity to noxious stimuli is present, and thus the perception of pain is exaggerated. Allodynia is a condition in which normally non-noxious stimuli become painful. Persistent or chronic pain, manifested as hyperalgesia and/or allodynia, remains challenging to treat. Many patients do not respond to existing therapeutics, or have their pain poorly managed (i.e., inadequate relief), or experience relief of an inadequate duration.

Endogenous reactive species produced by injury, irritant and disease are key drivers of pain as can be demonstrated in animal models of hyperalgesia and allodynia. Reactive oxygen species (ROS) and reactive nitrogen species (RNS) include free radicals such as superoxide and hydroxyl radical as well as the powerful oxidants peroxynitrite (OONO⁻), and (hydrogen) peroxide (H₂O₂). Both peroxynitrite (PN) and hydrogen peroxide, generated in the periphery after injury, contribute to changes in excitability in sensory afferents.

Peroxynitrite has been implicated in the development of opiate-induced antinociceptive (pain) tolerance (tachyphylaxis) (Muscoli et al., 2007, J Clin Invest 117:3530-3539). Peroxynitrite results from the diffusion-controlled reaction of superoxide (O₂⁻) and nitric oxide (NO). Unlike other endogenously produced reactive species/oxidants, peroxynitrite is not managed by enzymatic control. Peroxynitrite formation is facile, unleashing its powerful oxidative properties essentially unchecked, causing downstream effects that can cause pain.

In contrast, superoxide is formed from the action of NADPH oxidases and xanthine oxidase, and nitric oxide is produced by nitric oxide synthases (NOS). Hydrogen peroxide is formed from superoxide and the action of superoxide dismutase. During cellular stress (e.g., inflammation, nerve injury, ischemia), the action of these enzymatic systems can cause nitric oxide, superoxide and peroxide levels to increase significantly, which can lead to neuronal damage, hyperalgesia and allodynia. Concomitant increases in nitric oxide and superoxide can lead to greatly increased localized increases in peroxynitrite, which is capable of nitrating tyrosine residues within proteins, cross-linking cysteine residues and disrupting glutathione-disulfide homeostasis. Collectively, these effects lead to neuronal sensitization and pain, including neuropathic pain.

Diabetes is a leading cause of neuropathy. Approximately 50% of diabetic patients will develop peripheral neuropathy which manifests as burning, excruciating, stabbing or intractable types of pain. The currently available therapeutics are palliative, effective in only a portion of patients in providing symptomatic relief, and are not disease-modifying (diabetes). More troubling, even patients who initially experience relief from a given therapeutic usually revert to a painful state over time. Anticonvulsants such as pregabalin, gabapentin and lamotrigine and older tricyclic antidepressants (TCA) such as carbamazepine can be effective but are prone to produce CNS-associated adverse effects (e.g., sedation, cognitive deficits). Antidepressants belonging to the norepinephrine- and/or serotonin-reuptake inhibitors (SNRIs) class such as duloxetine are useful alternatives in some patients. The use of opioids and non-steroidal anti-inflammatory drugs (NSAIDs) is commonplace but not preferable due to abuse potential, withdrawal, tolerance leading to dose-escalation, constipation, nausea, vomiting and respiratory depression well-known to occur with opioid therapy and gastrointestinal ulceration and nephrotoxicity associated with NSAID usage. Lastly, topical agents (capsaicin, topical nitrates and topical TCAs) and local anesthetics have been used with mixed results.

Collectively, the treatment of painful diabetic neuropathy remains poorly managed as evident by Numbers-Needed-to-Treat values which range from 5 to 6 for the mostly widely used drugs (NEURONTIN®, LYRICA®, CYMBALTA®) (Treatment of Painful Diabetic Neuropathy-, Ther. Adv. Chronic Dis. 2015, 6 (1) 15 (S Javed).

There is a need in the medical and patient communities for a new class of therapeutic agents that can relieve a wide range of pain, including, but not limited to painful diabetic neuropathy. The methods and compounds described herein address this pressing need.

### SUMMARY OF THE INVENTION

In various embodiments, a method of treating diabetic neuropathy or symptoms associated with diabetic neuropathy in an individual is provided. The method includes administering a therapeutically effective amount of a composition comprising a compound of Formula I:

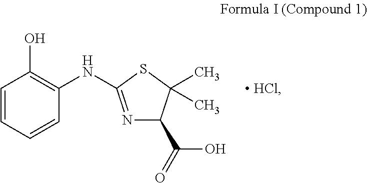 [SMILES: CC1(C)SC(Nc2ccccc2O)=N[C@@H]1C(=O)O]

to an individual having diabetic neuropathy or symptoms associated with diabetic neuropathy.

In various embodiments, a method of improving Quality-of-Life (QoL) such as range of mobility, level of physical activity and sleep quality in an individual having diabetic neuropathy or symptoms associated with diabetic neuropathy is provided. The method includes administering a therapeutically effective amount of a composition comprising a compound of Formula I:

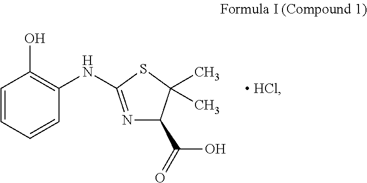 [SMILES: CC1(C)SC(Nc2ccccc2O)=N[C@@H]1C(=O)O]

to an individual having diabetic neuropathy or symptoms associated with diabetic neuropathy.

In various embodiments, Compound 1 advantageously has low toxicity, is an effective anti-hyperalgesic agent, and/or alleviates or prevents pain (analgesic effect). In various embodiments, administration of Compound 1 to an individual advantageously does not result in at least one of the following side effects: respiratory depression, constipation, nausea, vomiting, addiction, gastrointestinal ulceration or irritation, high blood pressure, low blood pressure, abdominal pain, arrhythmia, shortness of breath, fatigue, fainting, fluid build-up, reduced liver function, reduced renal function, inflammation, diarrhea, and/or tolerance to pain (anti-hyperalgesic effect).

### BRIEF DESCRIPTION OF THE FIGURES

The drawings illustrate generally, by way of example, but not by way of limitation, various embodiments of the present application.

FIG. 1 is an X-ray crystal structure of (R)-2-(2-hydroxyphenylamino)-5,5-dimethyl-4,5-dihydrothiazole-4-carboxylic acid mono-hydrochloride (Compound 1), in accordance with various embodiments.

FIG. 2 is an infrared (IR) spectrum of Compound 1, in accordance with various embodiments.

FIG. 3 is a ¹H-NMR (nuclear magnetic resonance) spectrum of Compound 1, in accordance with various embodiments.

FIG. 4 is a ¹³C-NMR spectrum of Compound 1, in accordance with various embodiments.

FIG. 5 is an experimental XRPD (X-ray powder diffraction) trace (bottom trace) and a calculated XRPD trace (top trace) for Compound 1, in accordance with various embodiments.

FIG. 6 is a Gravimetric Vapor Sorption (GVS)/Dynamic Vapor Sorption (DVS) isotherm plot for Compound 1, in accordance with various embodiments.

FIG. 7 is a combined DSC/TGA trace for Compound 1, in accordance with various embodiments.

FIG. 8 is a listing of structures of impurities potentially formed during the manufacture of Compound 1, in accordance with various embodiments.

FIG. 9 illustrates a non-limiting pathway for forming impurity Cmp1 Imp-3, which is potentially formed during the manufacture of Compound 1, in accordance with various embodiments.

FIG. 10 illustrates non-limiting reversal of mechanical hypersensitivity by Compound 1 in a diabetic neuropathy model, in accordance with various embodiments. Streptozotocin (STZ) destroys insulin-producing cells and generates a diabetic phenotype in mice. Animals were dosed with STZ on day −7. By day 0, animals are hyperglycemic and hyperalgesic (day 0 BL). Compound 1 blocks STZ-induced mechanical allodynia. Upon repeated dosing, Compound 1 is similar in efficacy and potency to gabapentin despite being peripherally restricted.

FIG. 11 illustrates an XPRD spectrum of amorphous Compound 1.

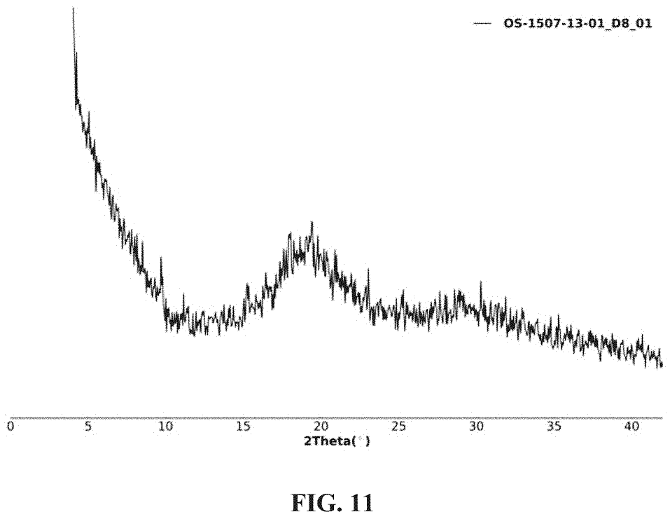

FIG. 12 illustrates a comparison of the XPRD spectra of Compound 1 free base (top trace) and Compound 1 (bottom trace).

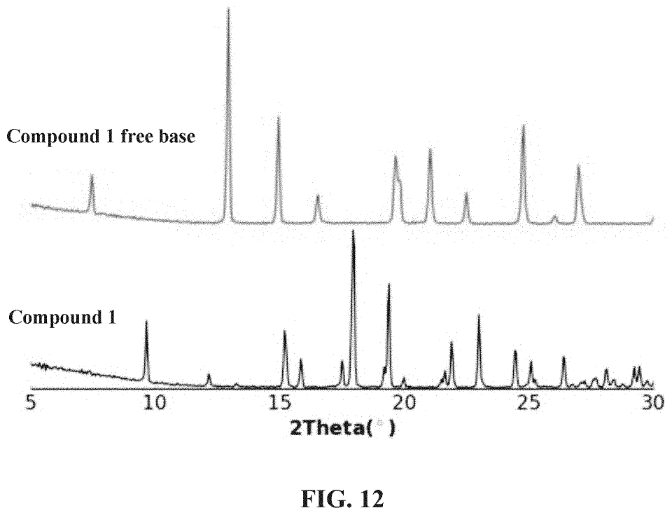

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used therein, “delaying” the development of post-surgical pain means to defer, hinder, slow, retard, stabilize, and/or postpone progression of post-surgical pain. This delay can be of varying lengths of time, depending on the history of the disease and/or individuals being treated. As is evident to one skilled in the art, a sufficient or significant delay can, in effect, encompass prevention, in that the individual does not develop post-surgical pain. A method that “delays” development of the symptom is a method that reduces probability of developing the symptom in a given time frame and/or reduces extent of the symptoms in a given time frame, when compared to not using the method.

As used herein, “development” or “progression” of post-surgical pain means initial manifestations and/or ensuing progression of the disorder. Development of post-surgical pain can be detectable and assessed using standard clinical techniques known in the art. However, “development” also refers to progression that may be undetectable. As used herein, “development” or “progression” refers to the biological course of the symptoms. “Development” includes occurrence, recurrence, and onset. As used herein “onset” or “occurrence” of post-surgical pain includes initial onset and/or recurrence.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, “mechanically-induced pain” or “mechanical hyperalgesia” refers to pain induced by a mechanical stimulus, such as the application of weight to a surface, tactile stimulus, and stimulation caused or associated with movement (including coughing, shifting of weight, etc.).

As used herein, “thermal hyperalgesia” refers to increased sensitivity to a thermal stimulus, such as the application warm or hot beam of light or a warm or hot surface such as a floor (plate) or a water or oil bath (immersion).

As defined herein, “nociceptive pain” refers to pain caused by activation of a specialized set of neurons called nociceptors which respond acutely to noxious stimuli. Common examples of harmful stimuli that cause nociceptive pain include damage to the skin, muscles, bones, or other tissues such as bruises, burns, fractures, cuts, scrapes, and the like.

As defined herein, “neuropathic pain” refers to pain caused by damage to neurons and the nervous system. Neuropathic pain is distinct from nociceptive pain, which is a ‘protective’ type of pain.

As defined herein, “diabetic neuropathy” means neuropathic pain experienced by individuals that have type I or type II diabetes. Neuropathic pain is associated with damage to the body's neurological system rather than damage to non-neuronal tissues as with nociceptive pain.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

Suitable pharmaceutically acceptable salts include zwitterions, compounds in which both a positive and negative charge exist on the same molecule.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, Pa.), which is incorporated herein by reference.

Other pharmaceutically acceptable excipients include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the compound(s) described herein are known in the art and described, for example in Genaro, ed. 1985, Remington's Pharmaceutical Sciences, Mack Publishing Co., Easton, Pa., which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human. The term “individual” as used herein, also refers to an individual or a subject, a patient or a person in need of relief of pain, or a human volunteer willing to be administered a therapeutic agent.

As used herein, the term “potency” refers to the dose needed to produce half the maximal response (ED₅₀).

As used herein, “palliating” means, in the context of post-surgical pain reducing the extent of one or more undesirable clinical manifestations of post-surgical pain in an individual or population of individuals treated with Compound 1.

As used herein, “reducing incidence” of pain means any of reducing severity (which can include reducing need for and/or amount of (e.g., exposure to) other drugs and/or therapies generally used for this conditions, including, for example, opiates), duration, and/or frequency (including, for example, delaying or increasing time to post-surgical pain in an individual). Since a patient's response to a treatment can vary, reducing incidence in the context of pain also means that there is a reasonable expectation by person of skill in the art that administration of Compound 1 is likely to result in a reduction in incidence of pain in a particular individual.

As used herein, “resting pain” refers to pain occurring even while the individual is at rest as opposed to, for example, pain occurring when the individual moves or is subjected to other mechanical stimuli (e.g., being moved in bed or out of bed, being helped to the bathroom, being moved in or out of a wheelchair, and the like).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds described herein (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics. Ameliorating post-surgical pain or one or more symptoms of post-surgical pain means a lessening or improvement of one or more symptoms of post-surgical pain after administration of Compound 1 as compared to not administering Compound 1, and also includes shortening or reduction in duration of a symptom.

The following abbreviations are used herein: BBr₃, boron tribromide; CD₃OD, (tetra)deuterio-methanol; COX, cyclooxygenase; d, day(s); DMSO, dimethylsulfoxide; DSC, differential scanning calorimetry; ELSD, evaporative light-scattering detection; g, gram; GC, gas chromatography; GC-MS, gas chromatography-mass spectrometry; GVS, gravimetric vapor sorption; h or hr, hour(s); HCl, hydrochloric acid; HPLC, high performance liquid chromatography; ICH, International Conference on Harmonisation; iPrOH, isopropanol; IR, infrared (spectrum); mg, milligram; m or min, minute(s); mL, milliliter; mol, mole; mmol, millimole; MTBE, methyl tert-butyl ether; NADPH, dihydronicotinamide-adenine dinucleotide phosphate; NaOH, sodium hydroxide; ng, nanogram; NLT, not less than; NMR, nuclear magnetic resonance; NMT, not more than; NOS, nitric oxide synthase; NSAID, non-steroidal anti-inflammatory drug; pKa, negative base-10 logarithm of the acid dissociated constant; PN, peroxynitrite; RNS, reactive nitrogen species; ROI, residue on ignition; ROS, reactive oxygen species; TRP, Transient-Receptor Potential; USP, United States Pharmacopeia; UV, ultraviolet; XRPD, x-ray (powder) diffraction pattern.

### Preparation of Compounds

The compound of Formula (I) can be prepared by the general schemes described herein, using the synthetic method known by those skilled in the art. The following examples illustrate non-limiting embodiments of the compound(s) described herein and their preparation.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, the compound(s) described herein can exist as zwitterions. All zwitterions are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4^{th} Ed., Vols. A and B (Plenum 2000, 2001), and Green & Wuts, Protective Groups in Organic Synthesis 3^{rd} Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

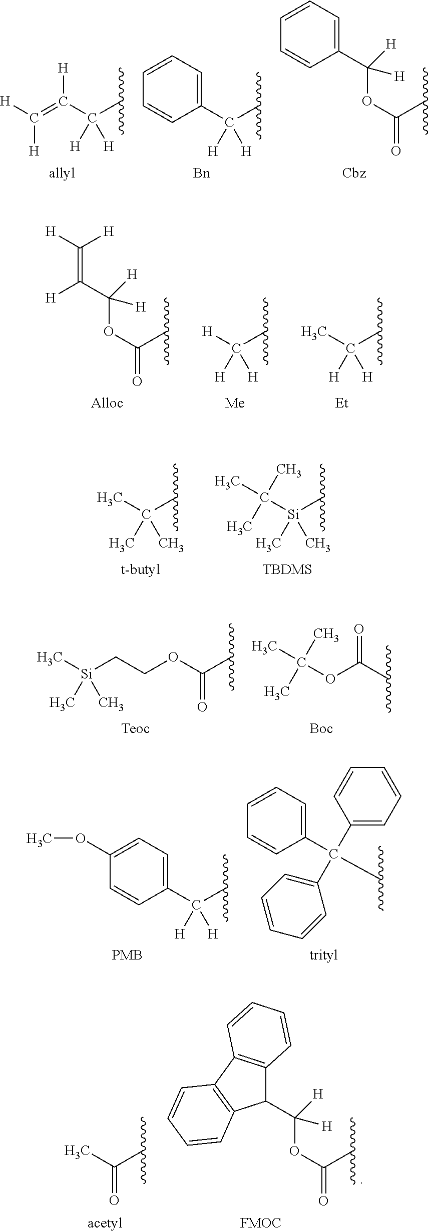

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

In various embodiments, a pharmaceutical composition of Compound 1 includes

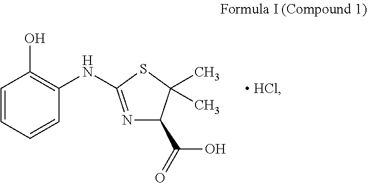 [SMILES: CC1(C)SC(Nc2ccccc2O)=N[C@@H]1C(=O)O]

and about 0.0001% to about 0.30% w/w of at least one impurity selected from the group consisting of 2-Cl-BO, BO-Imp-1, BO-Imp-2, BO-Imp-3, BO-Imp-4, BO-Imp-5, and Cmp1 Imp-3. The pharmaceutical composition can also include at least one pharmaceutically acceptable carrier, as described herein.

In various embodiments, the composition can include at least one pharmaceutically acceptable carrier and/or at least one pharmaceutically acceptable excipient. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences, 18^{th} Edition (1990, Mack Publication Co., New Jersey).

The composition can be prepared, packaged, or sold in the form of a sterile injectable aqueous or oily suspension or solution. This suspension or solution may be formulated according to the known art, and may comprise, in addition to the active ingredient, additional ingredients such as anti-oxidants, dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Compositions that are useful in the methods described herein can be administered, prepared, packaged, and/or sold in formulations suitable for intravenous, subcutaneous, sublingual, oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions can be administered via numerous routes, including, but not limited to, intravenous, subcutaneous, sublingual, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disorder being treated, the type and age of the veterinary or human patient being treated, and the like.

Compositions that are useful in the methods described herein can be administered systemically in intravenous and subcutaneous liquid formulations, oral and sublingual solid formulations, ophthalmic, suppository, aerosol, topical or other similar formulations. In addition to the compound such as heparin sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer compounds according to the methods as described herein.

The formulations of the compositions described herein can be prepared by any method known or hereafter developed in the art of pharmacology. In general, such preparatory methods include the step of bringing the active ingredient (e.g., Compound 1) into association with a carrier or one or more other accessory ingredients, and then, if necessary or desirable, shaping or packaging the product into a desired single- or multi-dose unit.

Although the descriptions of compositions provided herein are principally directed to pharmaceutical compositions which are suitable for ethical administration to humans, it will be understood by the skilled artisan that such compositions are generally suitable for administration to subjects of all sorts.

Modification of compositions suitable for administration to humans in order to render the compositions suitable for administration to various animals is well understood, and the ordinarily skilled veterinary pharmacologist can design and perform such modification with merely ordinary, if any, experimentation. Subjects to which administration of the compositions described herein are contemplated include, but are not limited to, humans and other primates, mammals including commercially relevant mammals such as cattle, pigs, horses, sheep, cats, and dogs.

Compositions that are useful in the methods described herein can be prepared, packaged, or sold in formulations suitable for intravenous, subcutaneous, sublingual, oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically based formulations.

A composition for use in the methods described herein can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage.

The relative amounts of the active ingredient (e.g., Compound 1), the pharmaceutically acceptable carrier, and any additional ingredients in a pharmaceutical composition described herein will vary, depending upon the identity, size, and condition of the subject treated and further depending upon the route by which the composition is to be administered. By way of example, the composition may comprise between 0.1% and 100% (w/w) active ingredient.

Liquid derivatives and natural extracts made directly from biological sources may be employed in the compositions described herein in a concentration (w/v) from about 1% to about 99%. Fractions of natural extracts and protease inhibitors may have a different preferred range, from about 0.01% to about 20% and, more preferably, from about 1% to about 10% of the composition. Of course, mixtures of the active agents described herein can be combined and used together in the same formulation, or in serial applications of different formulations.

The compositions described herein can include a preservative from about 0.005% to 2.0% by total weight of the composition. The preservative is used to prevent spoilage in the case of an aqueous gel because of repeated patient use when it is exposed to contaminants in the environment from, for example, exposure to air or the patient's skin, including contact with the fingers used for applying a composition described herein such as a therapeutic gel or cream. Examples of preservatives useful in accordance with the compound(s) described herein include but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition can include an antioxidant and a chelating agent which can inhibit any the degradation of Compound 1 that may occur, for use in an aqueous gel formulation. Suitable antioxidants include BHT, BHA, α-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Suitable dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Suitable emulsifying agents include, but are not limited to, lecithin, and acacia. Suitable preservatives include, but are not limited to, methyl, ethyl, or n-propyl-para-hydroxybenzoates, ascorbic acid, and sorbic acid. Suitable sweetening agents include, for example, glycerol, propylene glycol, sorbitol, sucrose, and saccharin. Suitable thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition(s) described herein can comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation(s) described herein can be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

The composition described herein can also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, an “oily” liquid is one which comprises a carbon-containing liquid molecule and which exhibits a less polar character than water.

A formulation of the compositions described herein suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The terms oral rinse and mouthwash are used interchangeably herein.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Compositions described herein can be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may include a powder or an aerosolized or atomized solution or suspension including the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

Typically, dosages of the compositions described herein can be administered to a subject, preferably a human, will vary depending upon any number of factors, including but not limited to, the type of animal and type of disease state being treated, the age of the subject and the route of administration.

### Method of Manufacturing

A method of making a compound of Formula I (Compound 1) is provided.

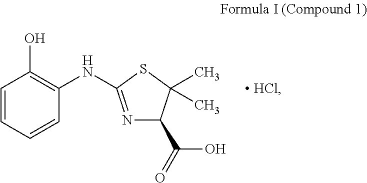 [SMILES: CC1(C)SC(Nc2ccccc2O)=N[C@@H]1C(=O)O]

The method includes reacting an amine compound with a structure of

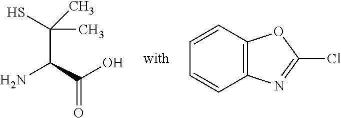

in the presence of a base and a first solvent to form an intermediate product of Formula II:

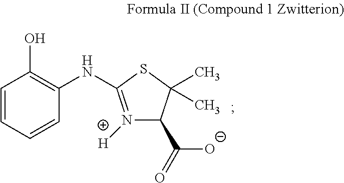

and
contacting the intermediate product with an acid and a second solvent to form Compound 1.

In various embodiments, Compound 1 can be prepared according to Scheme 1 as follows:

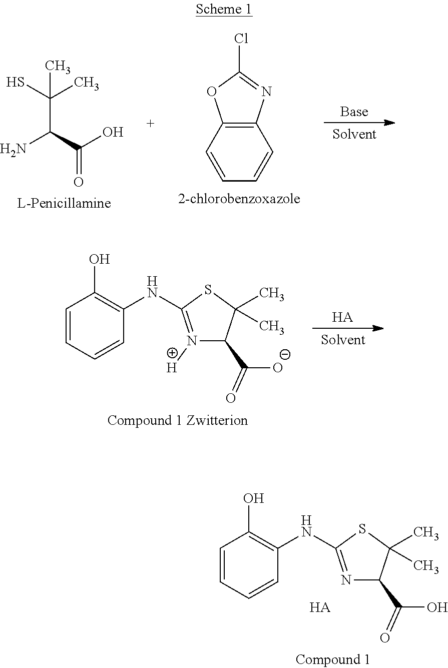

In various embodiments, Compound 1 Zwitterion is isolated prior to being treated with acid. The isolation can be carried out by methods known in the art such as re-crystallization or precipitation from a suitable solvent, such as iso-propanol, in which Compound 1 Zwitterion is insoluble or sparingly soluble.

Compound 1 Zwitterion can be prepared, in various embodiments, according to Scheme 2:

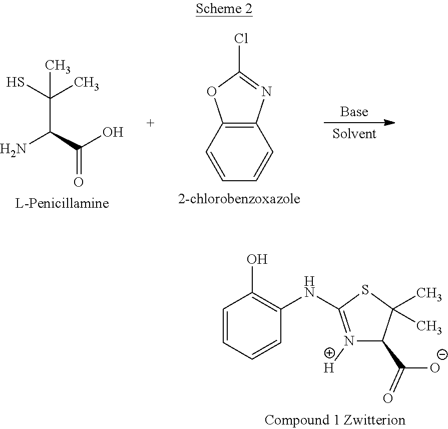

In various embodiments, isolated Compound 1 Zwitterion can be converted to Compound 1 according to Scheme 3:

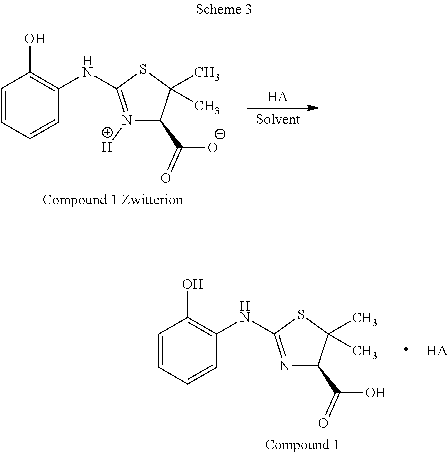

In Scheme 1 and Scheme 3, HA represents a protic acid, and A⁻ represents the conjugate base of HA.

The base in Scheme 1 can be any suitable base such as, without limitation, a primary, secondary, or tertiary amine, an alkyl lithium, a Grignard reagent, or an alkali metal hydroxide. In various embodiments, the base is selected from the group consisting of LiOH, NaOH, KOH, and combinations thereof. In various embodiments, the base is NaOH.

The first solvent can be any suitable solvent that is capable of dissolving the starting materials. The first solvent can be, in various embodiments, a polar protic solvent, a polar aprotic solvent, or any combination thereof. Suitable polar protic solvents can be, in various embodiments, water, methanol, ethanol, trifluoroethanol, iso-propanol, and mixtures thereof. In various embodiments, the polar aprotic solvent can be acetone, tetrahydrofuran, dimethylsulfoxide, acetonitrile, N,N-dimethylformamide, N-methyl-2-pyrrolidone, and mixtures thereof. The first solvent can also be a mixture of a protic polar solvent and an aprotic polar solvent, in any suitable ratio, such as from about 1:1 (protic:aprotic) to about 1:10 (protic:aprotic), or about 10:1 (protic:aprotic). In various embodiments, the first solvent is water.

The acid can be any suitable inorganic acid, such as HF, HCl, HBr, H₂SO₄, HNO₃, H₃NSO₃, H₃PO₄, and the like. The acid can also be an organic acid, such as acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like. In various embodiments, the acid is hydrochloric acid (HCl).

The second solvent can be any suitable solvent that is capable of dissolving polar substances such as Compound 1 Zwitterion. The second solvent can be, in various embodiments, a polar protic solvent, a polar aprotic solvent, or any combination thereof. Suitable polar protic solvents can be, in various embodiments, water, methanol, ethanol, trifluoroethanol, iso-propanol, and mixtures thereof. In various embodiments, the polar aprotic solvent can be acetone, tetrahydrofuran, dimethylsulfoxide, acetonitrile, N,N-dimethylformamide, N-methyl-2-pyrrolidone, and mixtures thereof. The second solvent can also be a mixture of a protic polar solvent and an aprotic polar solvent, in any suitable ratio, such as from about 1:1 (protic:aprotic) to about 1:10 (protic:aprotic), or about 10:1 (protic:aprotic). In various embodiments, the second solvent is iso-propanol.

Although Compound 1 is a hydrochloride acid addition salt, other pharmaceutically acceptable acid addition salts can be used in the methods described herein. “Pharmaceutically-acceptable acids” refers to those acids that are not toxic or otherwise biologically undesirable. Pharmaceutically acceptable acid addition salts can be formed with pharmaceutically acceptable inorganic acids including, but not limited to, hydrobromic acid, sulfuric acid, sulfamic acid, nitric acid, phosphoric acid, and the like.

Pharmaceutically acceptable acid addition salts can also be formed with pharmaceutically acceptable organic acids. Examples of pharmaceutically-acceptable organic acids, include but are not limited to, acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like. The methods can be used to economically scale the preparation of Compound 1 to commercial-scale operations if desired. The methods advantageously use inexpensive and environmentally benign reagents to produce Compound 1.

### Physical Properties of Compound 1

Compound 1, (R)-2-(2-hydroxyphenylamino)-5,5-dimethyl-4,5-dihydrothiazole-4-carboxylic acid mono-hydrochloride, has the structure of Formula I:

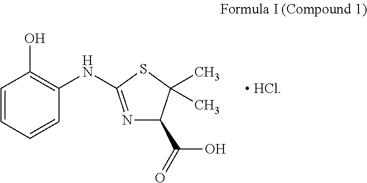 [SMILES: CC1(C)SC(Nc2ccccc2O)=N[C@@H]1C(=O)O]

Compound 1 has the following pKa values: 2.29±0.02 (Acidic), 6.97±0.01 (Basic), and 10.24±0.03 (Acidic). Compound 1 is freely soluble in methanol and tert-butyl alcohol:water (1:1). Compound 1 is sparingly soluble in iso-propanol, ethanol, 10% water:iso-propyl acetate, 10% water/tetrahydrofuran, and water. Compound 1 is less than sparingly soluble in n-heptane, toluene, acetone, tetrahydrofuran, ethyl acetate, iso-propyl acetate, tert-butyl methyl ether, and tert-butyl alcohol.

Compound 1 has a Log D distribution coefficient at pH 7.2 of −0.07 (3 mL PBS Buffer: 1 mL Octanol) and −0.39 (2 mL PBS Buffer: 2 mL Octanol), where PBS is phosphate buffer solution.

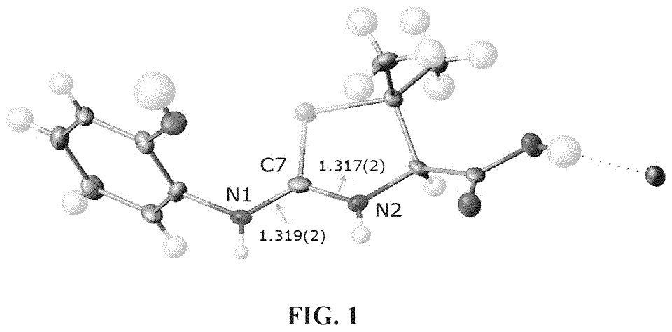

FIG. 1 shows the X-ray crystal structure of Compound 1. The crystallographic parameters for the structure in FIG. 1 are listed in Table 1 below.

TABLE 1
Crystal Data for (R)-2-(2-hydroxyphenylamino)-5,5-dimethyl-
4,5-dihydrothiazole-4-carboxylic acid mono-hydrochloride

| Crystal System | Orthorhombic | |
|---|---|---|
| Space Group | P212121 | |
| Unit Cell Dimensions | a = 7.00762(9) Å | α = 90° |
| | b = 10.08020 (10) Å | β = 90° |
| | c = 20.5203(2) Å | γ = 90° |

| | |
|---|---|
| | Volume = 1449.52(3) Å³ |
| Goodness of Fit on F² | 1.046 |
| Z′ | 4 |

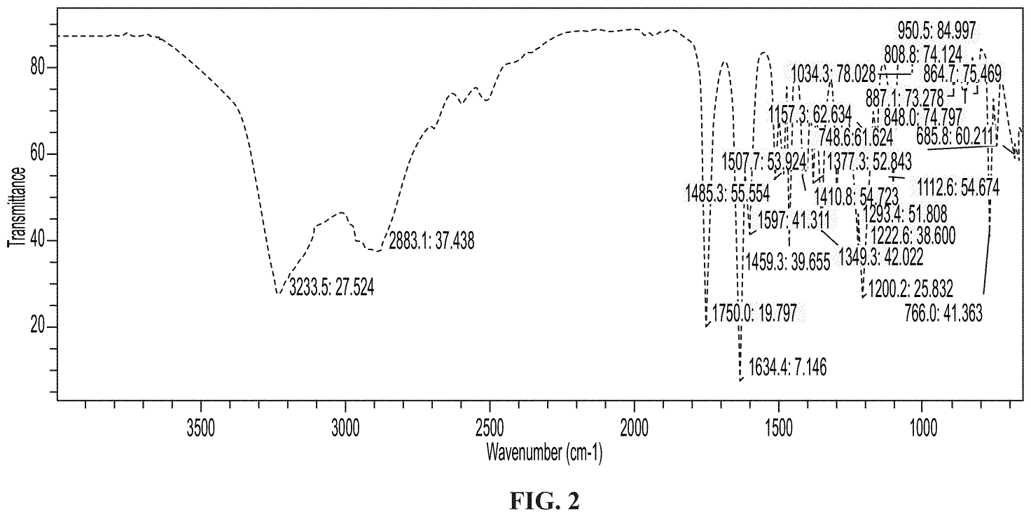

Table 2 lists the peak assignments of the functional groups in Compound observed in the infrared spectrum of Compound 1 (FIG. 2).

TABLE 2
Interpretation of (R)-2-(2-hydroxyphenylamino)-5,5-dimethyl-4,5-
dihydrothiazole-4-carboxylic acid mono-hydrochloride IR Data

| Range of | | | |
|---|---|---|---|
| Absorption | | | |
| (cm⁻¹) | Functional Group | Intensity | Type of Vibrations |
| *3200-3300 | N—H (Amine) | Broad | N—H Stretching |
| 2830-3000 | O—H (Acid) | Very broad | O—H Stretching |
| 1690-1750 | C═O (Carbonyl) | Sharp | C═O Stretching |
| 1590-1650 | C═N | Sharp | C═N Stretching |
| 1400-1600 | C═C | Medium | C═C Stretching |
| | | | (Aromatic) |

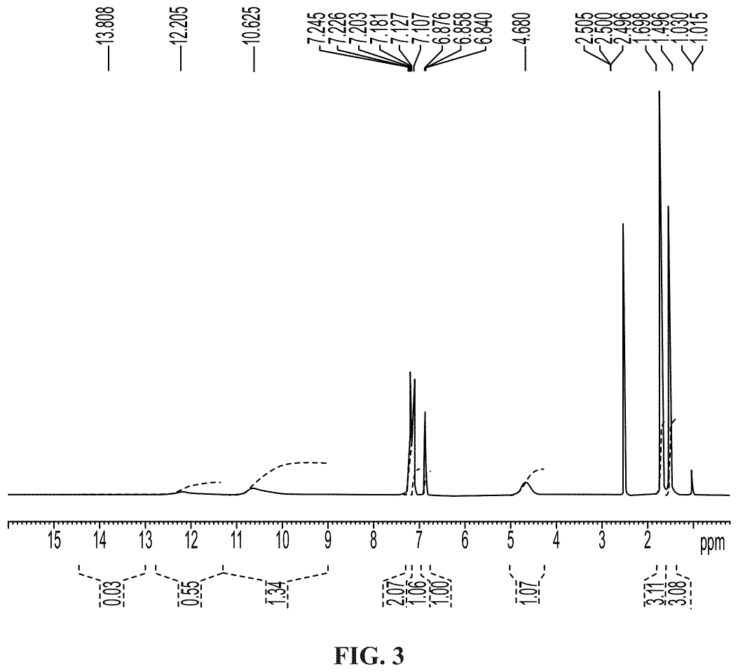

Table 3 lists the peak assignments for the hydrogen nuclei in the ¹H NMR spectrum of Compound 1 (FIG. 3).

TABLE 3
Interpretation of ¹H-NMR Spectrum of (R)-2-(2-hydroxyphenylamino)-
5,5-dimethyl-4,5-dihydrothiazole-4-carboxylic acid mono-hydrochloride

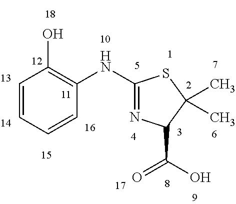

(R)-2-((2-hydroxyphenyl)amino)-5,5-dimethyl-4,5-
dihydrothiazole-4-carboxylic acid

| | | | Total Proton |
|---|---|---|---|
| Chemical Shift (ppm) | Multiplicity | Proton Number | Integration |
| 12.205 | Broad singlet | OH | 1 |
| 10.625 | Broad singlet | NH | 1 |
| 7.245 − 7.181 | multiplet | 14&16 | 2 |
| 7.127 − 7.107 (J = 8) | doublet | 13 | 1 |
| 6.876 − 6.840 (J = 7.2) | triplet | 15 | 1 |
| 4.680 | singlet | 3 | 1 |
| 1.698 | Singlet | 6 | 3 |
| 1.496 | Singlet | 7 | 3 |

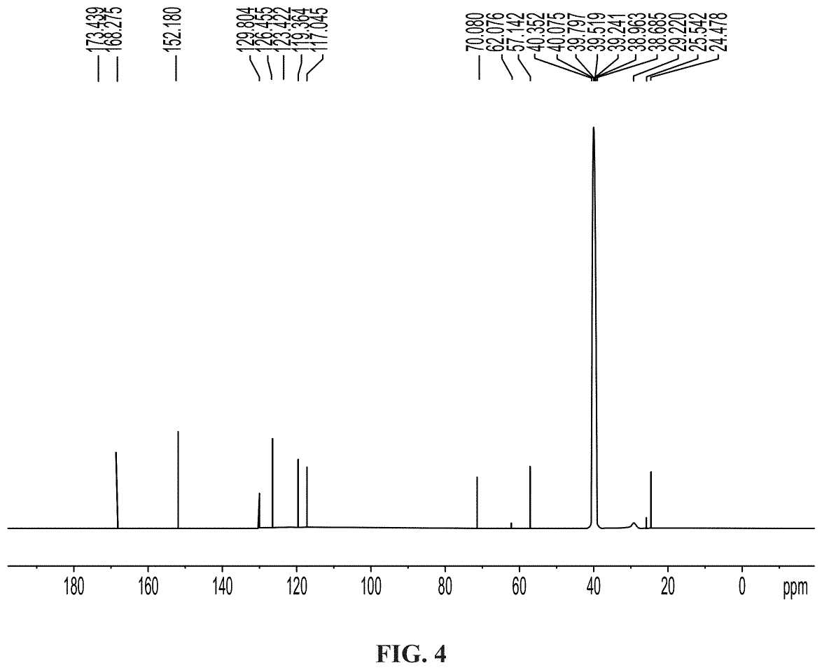

Table 4 lists the peak assignments for the carbon nuclei in the ¹³C NMR spectrum of Compound 1 (FIG. 4).

TABLE 4
Interpretation of ¹³C-NMR Spectrum of (R)-2-(2-hydroxyphenylamino)-
5,5-dimethyl-4,5-dihydrothiazole-4-carboxylic acid mono-hydrochloride

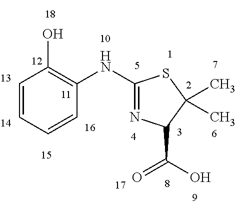

(R)-2-((2-hydroxyphenyl)amino)-5,5-dimethyl-4,5-dihydrothiazole-
4-carboxylic acid

| Chemical Shift (ppm) | Assignment | Number of Carbons | Type of Carbon |
|---|---|---|---|
| 24.48 | 6 | 1 | Primary |
| 29.22 | 7 | 1 | Primary |
| 57.14 | 2 | 1 | Quaternary |
| 70.98 | 3 | 1 | Tertiary |
| 117.05 | 13 | 1 | Tertiary |
| 119.36 | 15 | 1 | Tertiary |
| 123.42 | 11 | 1 | Quaternary |
| 126.46 | 16 | 1 | Tertiary |
| 129.80 | 14 | 1 | Tertiary |
| 152.18 | 12 | 1 | Quaternary |
| 168.28 | 8 | 1 | Quaternary |
| 173.44 | 5 | 1 | Quaternary |

Additional characteristics of Compound 1 and related compounds are described in U.S. Pat. No. 9,102,636, which is hereby incorporated by reference in its entirety.

### Polymorphs of Compound 1

Polymorphic screening of crystalline Compound 1 was performed using 15 organic/aqueous solvent systems, including: n-heptane, methanol, toluene, acetone, tetrahydrofuran, iso-propanol, ethanol, ethyl acetate, iso-propyl acetate, tert-butylmethyl ether, 10% water/90% iso-propyl alcohol, 10% water/90% tetrahydrofuran, tert-butyl alcohol, water, and 1:1 tert-butyl alcohol:water.

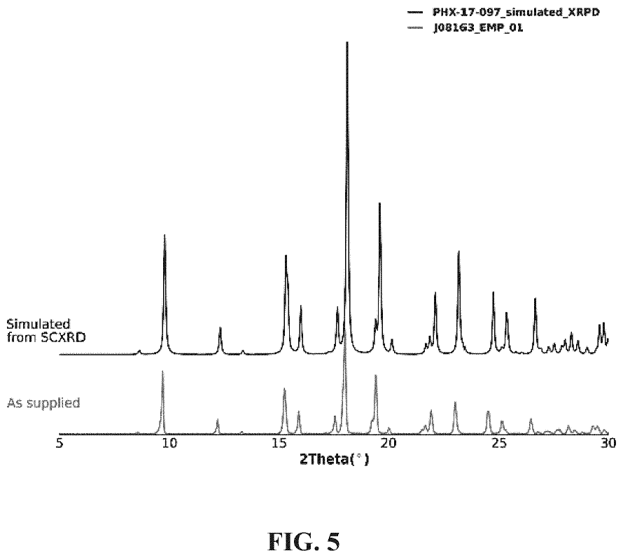

Only one crystalline form was obtained (Form 1). Compound 1 is a non-solvated, crystalline, mono-hydrochloride salt. FIG. 5 shows the experimentally obtained XPRD spectrum of Compound 1 in the bottom trace, and the simulated XPRD spectrum in the top trace. The XPRD spectrum was measured using Cu Kα radiation and collected from 2 to 42 degrees 2θ. The experimentally obtained XPRD spectrum of Compound 1 has the following peaks and associated intensities:

| Angle (2-Theta) | Intensity % |
|---|---|
| 9.6 | 43.3 |
| 12.2 | 10.7 |
| 13.3 | 4.5 |
| 15.2 | 37.6 |
| 15.8 | 19.9 |
| 17.5 | 18.7 |
| 18.0 | 100.0 |
| 19.2 | 14.8 |
| 19.4 | 66.6 |
| 20.0 | 8.3 |
| 21.5 | 7.2 |
| 21.7 | 12.6 |
| 21.9 | 31.0 |
| 23.0 | 47.6 |
| 24.5 | 25.2 |
| 25.1 | 18.6 |
| 25.2 | 6.9 |
| 26.4 | 21.2 |
| 26.7 | 4.1 |
| 27.1 | 5.4 |
| 27.2 | 6.4 |
| 27.7 | 8.1 |
| 28.1 | 13.2 |
| 28.4 | 6.7 |
| 28.8 | 4.1 |
| 29.2 | 15.1 |
| 29.4 | 15.1 |
| 29.7 | 6.0 |
| 30.1 | 12.3 |
| 30.5 | 12.2 |
| 31.1 | 13.8 |
| 31.4 | 26.6 |
| 31.9 | 11.4 |
| 32.8 | 7.6 |
| 34.0 | 15.5 |
| 34.5 | 7.5 |
| 35.1 | 4.8 |
| 35.4 | 6.6 |
| 35.7 | 5.0 |
| 36.4 | 6.9 |
| 36.9 | 3.8 |
| 37.5 | 13.8 |
| 37.7 | 8.3 |
| 38.0 | 4.8 |
| 38.5 | 6.6 |
| 39.0 | 5.6 |
| 39.3 | 15.5 |
| 39.7 | 3.1 |
| 40.3 | 5.1 |
| 40.6 | 5.4 |
| 40.7 | 5.3 |
| 41.5 | 6.7 |

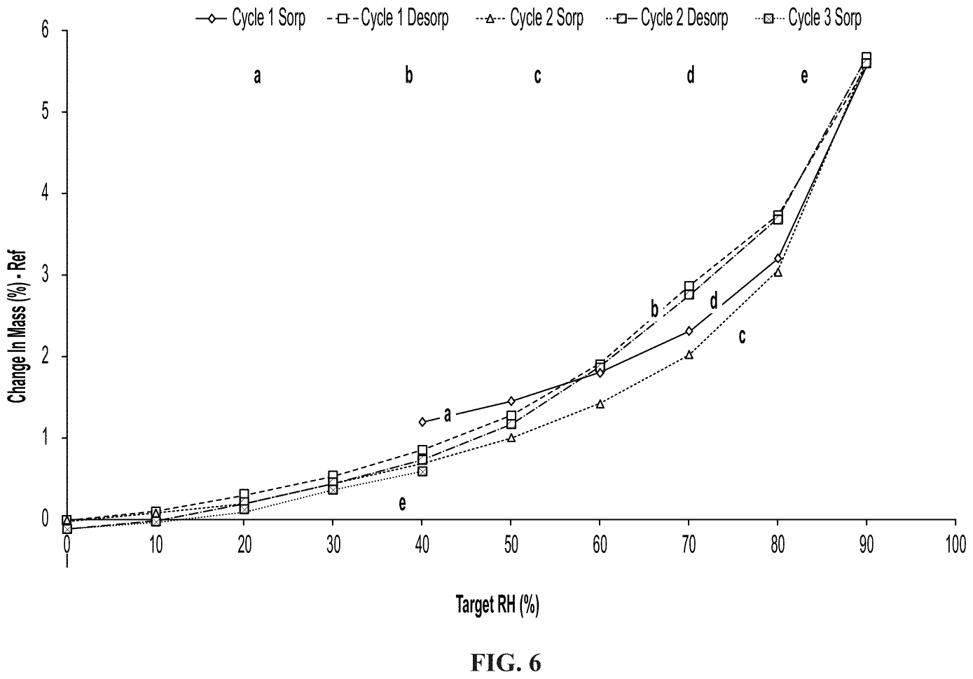

Gravimetric Vapor Sorption (GVS) shows an uptake of 6% between 0% and 90% RH. The sample is hygroscopic. The GVS isotherm plot is provided in FIG. 6.

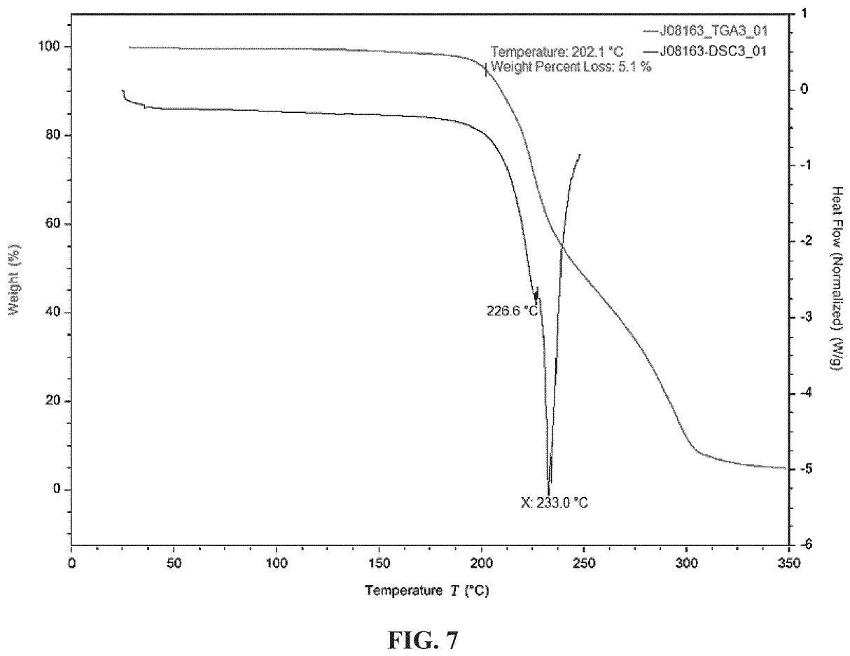

The combined DSC/TGA results for (R)-2-(2-hydroxyphenylamino)-5,5-dimethyl-4,5-dihydrothiazole-4-carboxylic acid mono-hydrochloride is provided in FIG. 7. The DSC shows a split endotherm between 200° C. and 250° C. and the TGA shows that decomposition (total 5% mass loss) starts at ˜202° C. An amorphous form of Compound 1 can be made by, for example, lyophilizing crystalline Compound 1 as described in Example 4 herein.

### Impurities in Compound 1

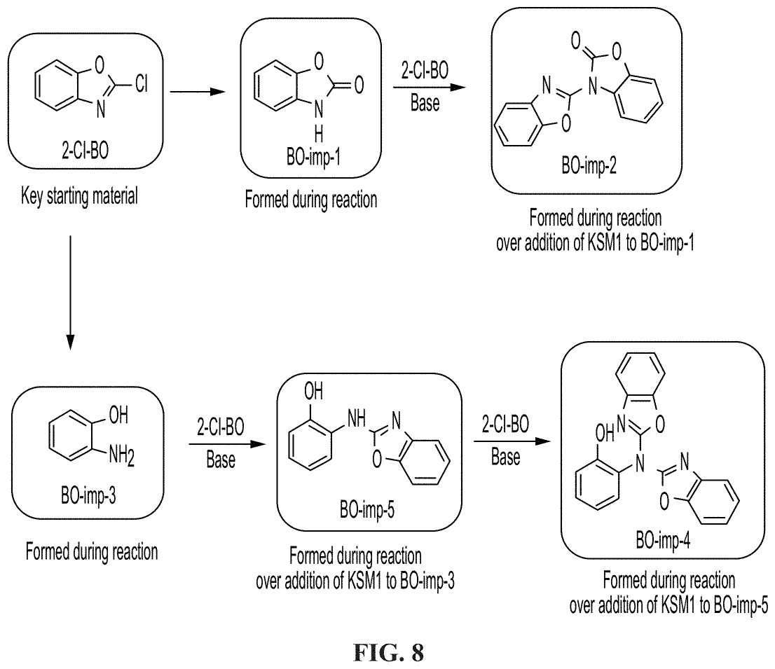

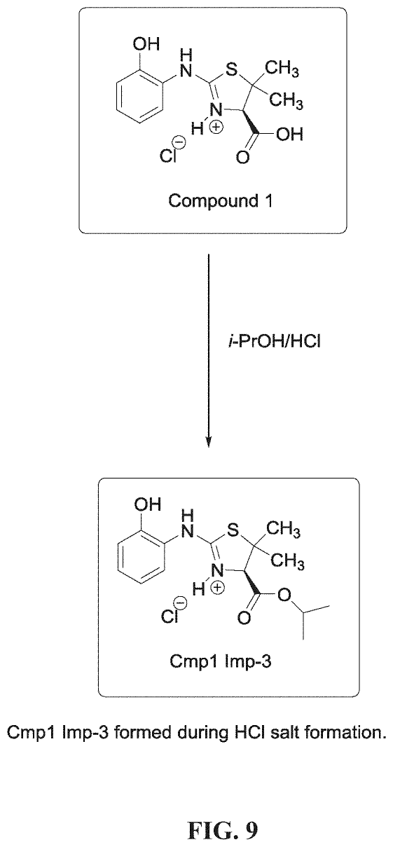

In various embodiments, Compound 1 described herein can include up to about 0.30% w/w of one or more impurities set forth in Table 5 below, and as shown in FIG. 8 and FIG. 9.

TABLE 5
Impurities in Compound 1

| Abbreviation | Chemical Name | Structure |
|---|---|---|
| 2-Cl-BO | 2-Chlorobenzoxazole | 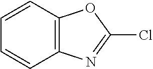 |
| L-Penicillamine | L-Penicillamine | 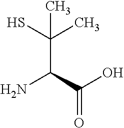 |
| BO-Imp-1 | 2-Hydroxybenzoxazole | 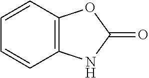 |
| BO-Imp-2 | 2'H-[2,3′-bi-1,3-benzoxazol]-2′-one | 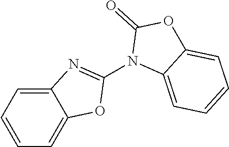 [SMILES: O=c1oc2ccccc2n1-c1nc2ccccc2o1] |
| BO-Imp-3 | 2-Aminophenol | 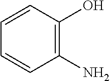 |
| BO-Imp-4 | 2-[Bis(1,3-benzoxazol-2-yl)amino]phenol | 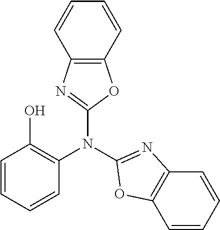 [SMILES: Oc1ccccc1N(c1nc2ccccc2o1)c1nc2ccccc2o1] |
| BO-Imp-5 | 2-[(1,3-Benzoxazol-2-yl)amino]phenol | 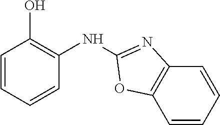 [SMILES: Oc1ccccc1Nc1nc2ccccc2o1] |
| Cmp1 Imp-3 | Propan-2-yl (4R)-2-(2- hydroxyanilino)-5,5-dimethyl-4,5- dihydro-1,3-thiazole-4-carboxylate | 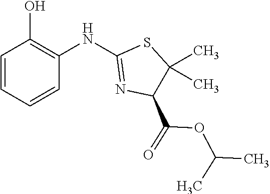 [SMILES: CC(C)OC(=O)[C@H]1N=C(Nc2ccccc2O)SC1(C)C] |

In various embodiments, Compound 1 has less than about 0.30% w/w, 0.25% w/w, 0.20% w/w, or 0.15% w/w of at least one impurity selected from the group consisting of 2-Cl-BO, BO-Imp-1, BO-Imp-2, BO-Imp-3, BO-Imp-4, BO-Imp-5, and Cmp1 Imp-3. In various embodiments, Compound 1 has about 0.0001% to about 0.30% w/w, about 0.0001% to about 0.25% w/w, about 0.0001% to about 0.20% w/w, about 0.001% to about 0.15% w/w, or about 0.01% to about 0.15% w/w of at least one impurity selected from the group consisting of 2-C1-BO, BO-Imp-1, BO-Imp-2, BO-Imp-3, BO-Imp-4, BO-Imp-5, and Cmp1 Imp-3.

In various embodiments, Compound 1 has about 0.0005%, 0.001%, 0.002%, 0.003%, 0.004%, 0.005%, 0.006%, 0.007%, 0.008%, 0.009%, 0.010%, 0.012%, 0.014%, 0.016%, 0.018%, 0.020%, 0.022%, 0.024%, 0.026%, 0.028%, 0.030%, 0.032%, 0.034%, 0.036%, 0.038%, 0.040%, 0.042%, 0.044%, 0.046%, 0.048%, or 0.050% w/w of at least one impurity selected from the group consisting of 2-Cl-BO, BO-Imp-1, BO-Imp-2, BO-Imp-3, BO-Imp-4, BO-Imp-5, and Cmp1 Imp-3. In various embodiments, Compound 1 includes about 0.010% to about 0.020% w/w of impurity BO-Imp-1 and about 0.002% to about 0.004% w/w of impurity BO-Imp-5. In various embodiments, one or more of the impurities in Compound 1 described herein are present in isolated Compound 1 in the amounts described herein. In various embodiments, one or more of the impurities in Compound 1 described herein are present in isolated and purified Compound 1 in the amounts described herein. A purified Compound 1 is a quantity of Compound 1 that was subjected to one or more of any of the analytical purification techniques described herein, or other purification techniques known in the art.

Impurities BO-Imp-1 through BO-Imp-5 can arise from the 2-chlorobenzoxazole starting material. A flow chart showing the formation of these impurities is provided in FIG. 8.

BO-Imp-3 is a process impurity which forms by hydrolysis of 2-chlorobenzoxazole by a minor competitive reaction pathway with sodium hydroxide. It can be purged by filtration of the zwitterion of Compound 1. BO-Imp-3 can form as a minor impurity (0.3%) during forced degradation testing of Compound 1, such with 5N sodium hydroxide heating for 5 h.

Cmp1 Imp-3 is a process impurity that forms via acid catalyzed esterification of salt-free Compound 1 with iso-propanol solvent during the hydrochloride salt formation. Its formation can be minimized by using stoichiometric hydrogen chloride in iso-propanol, which is added to a pre-cooled suspension of the zwitterion of Compound 1 in iso-propanol. It can be purged by filtration of Compound 1. Cmp1 Imp-3 is formed as shown in FIG. 9.

The enantiomer of Compound 1 is (S)-2-(2-hydroxyphenylamino)-5,5-dimethyl-4,5-dihydrothiazole-4-carboxylic acid mono-hydrochloride, and can be designated (S)-Compound 1. In various embodiments, the enantiomeric purity of Compound 1 can be at least about 95%, 97%, 98%, 99%, 99.2%, 99.4%, 99.6%, 98.8%, 99.9%, 99.99%, or more. Thus, for example, if the enantiomeric purity of Compound 1 is 99.5%, the composition contains 99.5% Compound 1 and 0.5% (S)-Compound 1. The enantiomeric purity refers only to the relative amounts of Compound 1 and (S)-Compound 1, and additional impurities may be present as described herein.

### Methods of Treatment

In various embodiments, a method of treating diabetic neuropathy is provided. The method includes administering a therapeutically effective amount of a composition that includes a compound of Formula I:

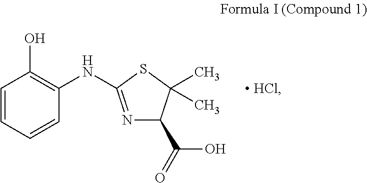 [SMILES: CC1(C)SC(Nc2ccccc2O)=N[C@@H]1C(=O)O]

to an individual having diabetic neuropathy or symptoms of diabetic neuropathy. Although Compound 1 is crystalline, the amorphous form of Compound 1 can also be used in the method of treating post-surgical pain described herein. In various embodiments, Compound 1 is the only pharmaceutically active agent in the composition. Additionally, a mixture of crystalline Compound 1 and amorphous Compound 1, in any proportions, can also be used in the method of treating post-surgical pain described herein.

The enantiomer of Compound 1 is (S)-2-(2-hydroxyphenylamino)-5,5-dimethyl-4,5-dihydrothiazole-4-carboxylic acid mono-hydrochloride, and can be designated (S)-Compound 1. In various embodiments, the enantiomeric purity of Compound 1 can be at least about 95%, 97%, 98%, 99%, 99.2%, 99.4%, 99.6%, 98.8%, 99.9%, 99.99%, or more. Thus, for example, if the enantiomeric purity of Compound 1 is 99.5%, the composition contains 99.5% Compound 1 and 0.5% (S)-Compound 1. The enantiomeric purity refers only to the relative amounts of Compound 1 and (S)-Compound 1, and additional impurities may be present as described herein. In various embodiments, the composition includes a therapeutically effective amount of a racemic mixture of Compound 1. A racemic mixture of Compound 1 contains about 50% Compound 1 and about 50% (S)-Compound 1.

Neuropathic pain is often considered a maladaptive chronic condition in which pain originates from damaged nerves, often yielding pain that is out-of-proportion to the extent of injury. The damage can occur from a physical injury such as trauma or from chemical injury as a result of, for example, chemotherapy. This type of neuropathic pain is an important component of a number of disease and/or disorders, with various sources, but that share the characteristic of development of a prolonged and profound pain state. Non-limiting examples of these diseases/disorders include spinal cord injury, post-herpetic neuralgia, diabetic neuropathy, phantom limb pain, stump/neuroma pain, post-ischemic pain (stroke), fibromyalgia, reflex sympathetic dystrophy (RSD), complex regional pain syndrome (CRPS), cancer-chemotherapeutic induced neuropathic pain, vertebral disk rupture, trigeminal neuralgia, and the like. In one embodiment, the disease or disorder is diabetic neuropathy.

In the context of treating symptoms of any of the neuropathies described herein, “reduce” means that the individual feels or suffers less from one or more of the symptoms than he or she would otherwise in the absence of treatment with Compound 1 or by treatment with another therapeutic agent. In the context of treating symptoms of any of the neuropathies described herein, “eliminate” means that the individual does not feel or notice one or more of the symptoms associated with particular neuropathy described herein.

The method can be used to treat peripheral neuropathy, which is the most common form of neuropathy linked to diabetes. Peripheral neuropathy, also called distal symmetric neuropathy or sensorimotor neuropathy. Peripheral neuropathy is often associated with damage to the nerves leading to an individual's feet, and can result in foot deformities, infections, ulcers, and amputations. In various embodiments, peripheral neuropathy includes nerve damage in the arms and/or legs. Symptoms of peripheral neuropathy may include numbness or insensitivity to pain or temperature; a tingling, burning, or prickling sensation; sharp pains or cramps; extreme sensitivity to touch, even light touch; loss of balance and coordination. The method can, in various embodiments, reduce or eliminate one or more of the symptoms of peripheral neuropathy described herein by administering a therapeutically effective amount of a composition containing Compound 1.

Peripheral neuropathy can also cause muscle weakness and loss of reflexes, especially at the ankle, leading to changes in a person's posture. In various embodiments, a method of reducing neurological effects of muscle weakness, loss of reflexes, or postural changes associated with peripheral neuropathy includes administering a therapeutically effective amount of a composition including Compound 1.

The method can also be used to treat proximal neuropathy, which is also called diabetic amyotrophy, lumbosacral plexus neuropathy, femoral neuropathy. This form of neuropathy specifically affects the muscles in the upper part of the leg(s), buttocks, and hips. Proximal neuropathy can also involve nerve pain, especially pain that shoots from the lower back and down the leg, which is called radiculopathy (sciatica). Proximal neuropathy causes weakness in the legs and difficulty or inability to stand from a sitting position without assistance. In such instances, treatment for weakness or pain is needed. Proximal neuropathy often affects elderly people with diabetes and subjects with type 2 diabetes. The method can, in various embodiments, reduce or eliminate one or more of the symptoms of proximal neuropathy described herein by administering a therapeutically effective amount of a composition containing Compound 1.

The method can also be used to treat autonomic neuropathy in a diabetic individual. Autonomic neuropathy affects the autonomic nerves responsible for maintaining unconscious bodily functions such as pumping of the heart, breathing, and digestion. Autonomic neuropathy can be particularly severe because it can affect many of the body's systems, including the digestive tract, vision, blood pressure blood glucose levels, respiratory function, urination, and sexual response. Symptoms of autonomic neuropathy include hypoglycemia unawareness (lack of symptoms when blood glucose levels drop below about 70 mg/dL), blood pressure drops as a result of damage to the nerves of the cardiovascular system, increased heart rate due to nerve damage associated with control of heart rate, gastroparesis (nerve damage to stomach), constipation (nerve damage to bowels), uncontrolled diarrhea (nerve damage to bowels), urinary tract infections, urinary incontinence, decreased sexual response in men and women, poor temperature regulation (damage to nerves that control sweating), and light insensitivity in the form of not being able to see well at night (damage to optic nerves). Proximal neuropathy often affects elderly people with diabetes and subjects with type 2 diabetes. The method can, in various embodiments, reduce or eliminate one or more of the symptoms of autonmic neuropathy described herein by administering a therapeutically effective amount of a composition containing Compound 1.

The method can also be used to treat focal neuropathy in a diabetic individual. Focal neuropathy affects a specific nerve rather than many nerves. Focal neuropathy, which often comes on suddenly, most often affects nerves in the head (especially nerves that connect to the eyes), although it can also affect the torso and legs. When focal neuropathy can affect the legs, it has different symptoms than proximal neuropathy. Proximal neuropathy causes muscle weakness in the legs, and it can also cause shooting pain down the leg. Focal neuropathy, however, causes pain in very specific locations on the legs.

Additionally, focal neuropathy can cause a variety of ailments, non-limiting examples of which include inability to focus the eye, double vision, aching behind one eye, paralysis on one side of the face (Bell's palsy), pain in the lower back or pelvis, pain in the front of a thigh, pain in the chest, stomach, or side, pain on the outside of the shin or inside of the foot, and pain in the chest or abdomen. Additionally, subjects with diabetes can develop nerve compressions, also called entrapment syndromes. The most common nerve compression is carpal tunnel syndrome, which causes numbness and tingling of the hand and sometimes muscle weakness or pain. Other nerves susceptible to entrapment can cause pain on the outside of the shin or the inside of the foot. The method can, in various embodiments, reduce or eliminate one or more of the symptoms of focal neuropathy described herein by administering a therapeutically effective amount of a composition containing Compound 1.

In some instances, the symptoms of any of the neuropathies both described herein and known in the art, including diabetic neuropathy, peripheral neuropathy, proximal neuropathy, focal neuropathy, and autonomic neuropathy are more intense at night. In various embodiments, administering Compound 1 or a composition thereof can improve an individual's sleep quality by reducing the intensity of or eliminating symptoms of peripheral neuropathy, proximal neuropathy, focal neuropathy, and/or autonomic neuropathy at night. Sleep quality can be measured by, for example, the protocols set forth in Krystal, A D, Edinger J D Sleep Med. 2008 September; 9 Suppl 1:S10-7 or in Landry, G. J., Best, J. R., and Liu-Ambrose, T. Front. Aging Neurosci. 2015; 7: 166. Sleep quality can also be assessed by less formal methods, such as by various smart watch devices (e.g., Apple® Watch or FitBit®), and the various measurements and assessments used in these devices or software applications running on such devices. In some embodiments, administration of Compound 1 can increase the amount of quality sleep experienced by an individual as assessed by the protocols described herein. by an individual as assessed by the protocols described herein. In various embodiments, administration of Compound 1 can increase the amount of “good” sleep as determined by a smart watch device or software application running on such a device.

Administration of Compound 1 to improve sleep quality can be at any suitable time prior to commencement of a sleep period. This period usually occurs at night, but the administration of Compound 1 is not limited to regularly scheduled sleep, and includes administration prior to engaging in any sleep or rest period lasting longer than about 30 minutes at any time of the day or night. In some embodiments, Compound 1 is administered about 15, 30, 45, 60, 75, 90, 105, 120, 135, or 150 minutes prior to the start of the intended sleep period or rest.

The method can also be used to treat pain occurring during progression of neurodegenerative diseases such as amyotrophic lateral sclerosis (ALS), Parkinson's Disease, Multiple Sclerosis, as well as neurotraumatic events such as stroke and ischemia.

Diagnosis or assessment of pain is well-established in the art. Assessment may be performed based on objective and/or subjective measure, such as observation of behavior such as reaction to stimuli, facial expressions and the like. Assessment may also be based on subjective measures, such as patient characterization of pain using various pain scales. See, e.g., Katz et al, Surg Clin North Am. (1999) 79 (2):231-52; Caraceni et al. J Pain Symptom Manage (2002) 23(3):239-55.

Pain relief can be characterized by time course of relief. Accordingly, in some embodiments, pain relief is subjectively or objectively observed after at least, greater than, or less than about 5 min, 10 min, 15 min, 20 min, 25 min, 30 min, 35 min, 40 min, 45 min, 50 min, 55 min, 60 min, 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h.

### Dosing and Dosing Regimens for Treatment of Diabetic Neuropathy

In various embodiments, the therapeutically effective amount of Compound 1 for treating diabetic neuropathy can be from about 5 mg to about 5000 mg. The therapeutically effective amount of Compound 1 can be about 10 mg to about 4750 mg, about 25 mg to about 4500 mg, about 50 mg to about 4250 mg, about 100 mg to about 4000 mg, about 150 mg to about 3750 mg, about 200 mg to about 3500 mg, about 275 mg to about 3250 mg, or about 100 mg to about 3000 mg, about 200 mg to about 2000 mg, or about 300 mg to 1000 mg. In various embodiments, the therapeutically effective amount of Compound 1 can be at least, equal to, or greater than about 5 mg, 10 mg, 20 mg, 40 mg, 60 mg, 80 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, 400 mg, 420 mg, 440 mg, 460 mg, 480 mg, 500 mg, 600 mg, 750 mg, 1000 mg, 1250 mg, 1500 mg, 1750 mg, 2000 mg, 2500 mg and 3000 mg.

The therapeutically effective amount of Compound 1 can be administered once a day, twice a day, three times a day, four times a day, or more. In various embodiments, the therapeutically effective amount of Compound 1 is administered for about 1 day to about 90 days. The therapeutically effective amount of Compound 1 can be administered for about 1 day, 2 days, 3 days, 4 days, 5 days, 6 days, 7 days, 14 days, 28 days, or more. Administering of Compound 1 can continue for as long as the individual, in consultation with a physician, deems it necessary to maintain adequate pain control for their individual situation. In various embodiments, Compound 1 can be administered for about 1 month to about 24 months, or for the lifespan of the individual.

In various embodiments, administering Compound 1 under any of the conditions described herein can result in a maximum observed plasma concentration (Cₘₐₓ) of about 5 μg/mL to about 300 μg/mL in a rat, mouse, dog, or human. The Cₘₐₓ of Compound 1 can be about 10 μg/mL to about 280 μg/mL, about 20 μg/mL to about 260 μg/mL, about 40 μg/mL to about 240 μg/mL, about 50 μg/mL to about 220 μg/mL, about 60 μg/mL to about 200 μg/mL, about 70 μg/mL to about 180 μg/mL, about 80 μg/mL to about 160 μg/mL, about 90 μg/mL to about 140 μg/mL, or about 95 μg/mL to about 120 μg/mL. In various embodiments, the Cₘₐₓ of Compound 1 can be at least, equal to, or greater than about 5 μg/mL, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 120 μg/mL, 140 μg/mL, 160 μg/mL, 180 μg/mL, 200 μg/mL, 220 μg/mL, 240 μg/mL, 260 μg/mL, 280 μg/mL, or about 300 μg/mL.

In various embodiments, administering Compound 1 under any of the conditions described herein can result in an area under the curve (AUC_{INF}) of about 100 hr·μg/mL to about 3000 hr·μg/mL in a rat, mouse, dog, or human. The AUC_{INF} of Compound 1 can be about 100 hr·μg/mL to about 2800 hr·μg/mL, about 200 hr·μg/mL to about 2600 hr·μg/mL, about 400 hr·μg/mL to about 2400 hr·μg/mL, about 500 hr·μg/mL to about 2200 hr·μg/mL, about 600 hr·μg/mL to about 2000 hr·μg/mL, about 700 hr·μg/mL to about 1800 hr·μg/mL, about 800 hr·μg/mL to about 1600 hr·μg/mL, about 900 hr·μg/mL to about 1400 hr·μg/mL, or about 950 hr·μg/mL to about 1200 hr·μg/mL. In various embodiments, the AUC_{INF} of Compound 1 can be at least, equal to, or greater than about 50 hr·μg/mL, 100 hr·μg/mL, 200 hr·μg/mL, 300 hr·μg/mL, 400 hr·μg/mL, 500 hr·μg/mL, 600 hr·μg/mL, 700 hr·μg/mL, 800 hr·μg/mL, 900 hr·μg/mL, 1000 hr·μg/mL, 1200 hr·μg/mL, 1400 hr·μg/mL, 1600 hr·μg/mL, 1800 hr·μg/mL, 2000 hr·μg/mL, 2200 hr·μg/mL, 2400 hr·μg/mL, 2600 hr·μg/mL, 2800 hr·μg/mL, or about 3000 hr·μg/mL.

The methods described herein can include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that reduces or ameliorates pain.

In certain embodiments, administering the compound(s) described herein to the subject allows for administering a lower dose of the additional therapeutic agent as compared to the dose of the additional therapeutic agent alone that is required to achieve similar results in treating, preventing, or ameliorating pain in the subject. For example, in certain embodiments, the compound(s) described herein enhances the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

In various embodiments, the method includes administering a therapeutically effective amount of a composition containing Compound 1 in combination or adjunctively with at least one additional pharmaceutically active agent. The type of pharmaceutically active agent that can be administered in combination or adjunctively with Compound 1 is not particularly limited. Non-limiting examples of additional pharmaceutically active agents include acetaminophen, alpha-2 adrenergic agonists, aspirin, COX-1 inhibitors, COX-2 inhibitors, voltage-gated ion channel blockers (NaV, CaV and KaV families), ligand-gated ion channels (TRPV1, TRPV4, TRPA1, and TRPM8 antagonists and agonists), opioid analgesics (mu-, delta-, kappa-selective and mixed), non-opioid analgesics, non-steroidal anti-inflammatories, norepinephrine reuptake inhibitors, serotonin reuptake inhibitors, dual norepinephrine-serotonin reuptake inhibitors, anticonvulsants (lamotrigine) including the gabapentinoids (gabapentin, pregabalin, mirogabalin), antidepressants (including tricyclics such as amitriptyline, doxepin and desipramine), tramadol and tapentadol.

Non-limiting examples of analgesic drugs that can be useful in combination or adjunctive therapy with Compound 1 include without limitation acetaminophen, alfentanil, allylprodine, alphaprodine, anileridine, aspirin, benzylmorphine, bezitramide, buprenorphine, butorphanol, clonidine, clonitazene, codeine, cyclazocine, desomorphine, dextromoramide, dextropropoxyphene, dezocine, diampromide, diamorphone, dihydrocodeine, dihydromorphine, dimenoxadol, dimepheptanol, dimethylthiambutene, dioxaphetyl butyrate, dipipanone, duloxetine, eptazocine, ethoheptazine, ethylmethylthiambutene, ethylmorphine, etonitazene, fentanyl, gabapentin, heroin, hydrocodone, hydromorphone, hydroxypethidine, isomethadone, ketobemidone, levallorphan, levorphanol, levophenacyl-morphan, lofentanil, meperidine, meptazinol, metazocine, methadone, metopon, mirogabalin, morphine, myrophine, nalbuphine, nalorphine, narceine, nicomorphine, norlevorphanol, normethadone, normorphine, norpipanone, opium, oxycodone, oxymorphone, papaveretum, pentazocine, phenadoxone, phenazocine, phenomorphan, phenoperidine, piminodine, piritramide, pregabalin, proheptazine, promedol, properidine, propiram, propoxyphene, sufentanil, tapentadol, tilidine, tramadol, NO-naproxen, NCX-701, ALGRX-4975, pharmaceutically acceptable salts thereof, and any combinations thereof.

Non-limiting examples of anticonvulsants that can be useful in combination or adjunctively with Compound 1 include without limitation acetylpheneturide, albutoin, aminoglutethimide, 4-amino-3-hydroxybutyric acid, atrolactamide, beclamide, buramate, carbamazepine, cinromide, clomethiazole, clonazepam, decimemide, diethadione, dimethadione, doxenitoin, eterobarb, ethadione, ethosuximide, ethotoin, felbamate, fluoresone, fosphenyloin, gabapentin, ganaxolone, lamotrigine, levetiracetam, lorazepam, mephenyloin, mephobarbital, metharbital, methetoin, methsuximide, midazolam, mirogabalin, narcobarbital, nitrazepam, oxcarbazepine, paramethadione, phenacemide, phenetharbital, pheneturide, phenobarbital, phensuximide, phenylmethylbarbituric acid, phenyloin, phenethylate, pregabalin, primidone, progabide, remacemide, rufinamide, suclofenide, sulthiame, talampanel, tetrantoin, tiagabine, topiramate, trimethadione, valproic acid, valpromide, vigabatrin, zonisamide, pharmaceutically acceptable salts thereof, and any combinations thereof.

Non-limiting examples of antidepressants that can be useful in combination or adjunctively with Compound 1 include without limitation bicyclic, tricyclic and tetracyclic antidepressants, hydrazides, hydrazines, phenyloxazolidinones and pyrrolidones. Specific examples include adinazolam, adrafinil, amineptine, amitriptyline, amitriptylinoxide, amoxapine, befloxatone, bupropion, butacetin, butriptyline, caroxazone, citalopram, clomipramine, cotinine, demexiptiline, desipramine, dibenzepin, dimetacrine, dimethazan, dioxadrol, dothiepin, doxepin, duloxetine, etoperidone, femoxetine, fencamine, fenpentadiol, fluacizine, fluoxetine, fluvoxamine, hematoporphyrin, hypericin, imipramine, imipramine N-oxide, indalpine, indeloxazine, iprindole, iproclozide, iproniazid, isocarboxazid, levophacetoperane, lofepramine, maprotiline, medifoxamine, melitracen, metapramine, metralindole, mianserin, milnacipran, minaprine, mirtazapine, moclobemide, nefazodone, nefopam, nialamide, nomifensine, nortriptyline, noxiptilin, octamoxin, opipramol, oxaflozane, oxitriptan, oxypertine, paroxetine, phenelzine, piberaline, pizotyline, prolintane, propizepine, protriptyline, pyrisuccideanol, quinupramine, reboxetine, ritanserin, roxindole, rubidium chloride, sertraline, sulpiride, tandospirone, thiazesim, thozalinone, tianeptine, tofenacin, toloxatone, tranylcypromine, trazodone, trimipramine, tryptophan, venlafaxine, viloxazine, zimeldine, pharmaceutically acceptable salts thereof, and any combinations thereof.

The additional pharmaceutically active agent can be included with Compound 1 in the same dosage form or in a separate dosage form, and any of the dosage forms described herein can be suitably used for combining Compound 1 and an additional pharmaceutically active agent in the same dosage form. When the additional pharmaceutically active agent is present in a separate dosage form, the additional pharmaceutically active agent can be administered at the same time as Compound 1 or at a different time, such as about 1 hour to about 24 hours after administration of Compound 1. The additional pharmaceutically active agent can be administered for the entire duration of administration of Compound 1, or for a shorter or longer time.

### Administration/Dosage/Formulations

The regimen of administration may affect what constitutes an effective amount. The therapeutic formulations may be administered to the subject either prior to or after the onset of pain. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat pain in the patient. An effective amount of the therapeutic compound necessary to achieve a therapeutic effect may vary according to factors such as the state of the disease or disorder in the patient; the age, sex, and weight of the patient; and the ability of the therapeutic compound to treat pain in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. “Dosage unit form,” as used herein, refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound for the treatment of pain in a patient.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of pain in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes Compound 1 can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be uncoated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing Compound 1. The coating can contain, for example, EUDRAGIT® L, S, FS, and/or E polymers with acidic or alkaline groups to allow release of Compound 1 in a particular location, including in any desired section(s) of the intestine. The coating can also contain, for example, EUDRAGIT® RL and/or RS polymers with cationic or neutral groups to allow for time-controlled release of Compound 1 by pH-independent swelling.

Hard capsules that include Compound 1 can be made using a physiologically degradable composition, such as gelatin. Such hard capsules include Compound 1, and can further include additional ingredients including, for example, an inert solid diluent such as calcium carbonate, calcium phosphate, or kaolin.

Soft gelatin capsules that include Compound 1 can be made using a physiologically degradable composition, such as gelatin. Such soft capsules include Compound 1, which may be mixed with water or an oil medium such as peanut oil, liquid paraffin, or olive oil.

Liquid formulations of compositions described herein which are suitable for oral administration can be prepared, packaged, and sold either in liquid form or in the form of a dry product intended for reconstitution with water or another suitable vehicle prior to use.

Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, and kidney dialytic infusion techniques.

For parenteral administration, the compound(s) described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Formulations of a pharmaceutical composition suitable for parenteral administration include the active ingredient (e.g. Compound 1) combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as antioxidants, dispersing agents, wetting agents, or suspending agents described herein. Such sterile injectable formulations can be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

In various embodiments, Compound 1 is administered to a patient by intravenous infusion over time. The dose of Compound 1 administered to the patient can be readily determined by a physician based on art-recognized patient characteristics as described herein. In various embodiments, the concentration of Compound 1 in the infusion is from about 0.01 mg/mL to about 3000 mg/mL. The volume of the infusion administered to the patient can be from about 1 mL to about 2000 mL in a given 24-h period. The intravenous infusion can be over any period as determined by the particular needs of the patient and their medical condition, which can readily be determined by a physician. In various embodiments, Compound 1 is administered over about 1 min to about 60 min, or over about 1 h to about 24 h. Suitable devices for use with intravenous infusion of Compound 1 include the BD Alaris™ Pump Module, the Alaris™ Syringe Module, and the like. Compound 1 can also be administered using a PCA (patient-controlled analgesia) device, wherein the patient controls when a bolus of the infusion containing Compound 1 is delivered. The volume of the bolus and the concentration of Compound 1 in the bolus can be any of the amounts described herein with respect to intravenous infusion. Suitable PCA devices for PCA delivery of Compound 1 include the Alaris™ PCA Module, and the like. Additional fluids, including other medications, saline solution, electrolytes, nutrition fluids (e.g., total parenteral nutrition), blood, and blood products, can be administered to the patient consecutively or concurrently with the intravenous infusion of Compound 1.

Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Enhancers of permeation can be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, polar lipids, or N-methyl-2-pyrrolidone.

One acceptable vehicle for topical delivery of some of the compositions described herein may contain liposomes. The composition of the liposomes and their use are known in the art (for example, see U.S. Pat. No. 6,323,219).

A topical dosage form of the inventive compound(s) can be optionally combined with other ingredients such as adjuvants, anti-oxidants, chelating agents, surfactants, foaming agents, wetting agents, emulsifying agents, viscosifiers, buffering agents, preservatives, and the like. In various embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

A topical dosage form of the inventive compound(s) should be applied in an amount effective to affect desired changes. As used herein “amount effective” shall mean an amount sufficient to cover the region of skin surface where a change is desired. In various embodiments, Compound 1 can be present in the amount of from about 0.0001% to about 15% by weight volume of the composition. In various embodiments, Compound 1 can be present in an amount from about 0.0005% to about 5% of the composition; most preferably, it should be present in an amount of from about 0.001% to about 1% of the composition.

Rectal Administration

Compositions described herein can be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining Compound 1 with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation.

The term, “delayed release,” is used herein in its conventional sense to refer to a drug formulation that provides for an initial release of the drug after some delay following drug administration and that mat, although not necessarily, includes a delay of from about 10 minutes up to about 12 hours.

The term, “pulsatile release,” is used herein in its conventional sense to refer to a drug formulation that provides release of the drug in such a way as to produce pulsed plasma profiles of the drug after drug administration.

The term, “immediate release,” is used in its conventional sense to refer to a drug formulation that provides for release of the drug immediately after drug administration.

As used herein, “short-term” refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, “rapid-offset” refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of pain in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term, “unit dosage form,” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized. Those skilled in the art will recognize, or be able to ascertain using no more than routine experimentation, numerous equivalents to the specific procedures, embodiments, claims, and examples described herein. Such equivalents were considered to be within the scope of this application and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### EXAMPLES

Various embodiments of the present application can be better understood by reference to the following Examples which are offered by way of illustration. The scope of the present application is not limited to the Examples given herein.

### Example 1: Preparation of Compound 1 Zwitterion

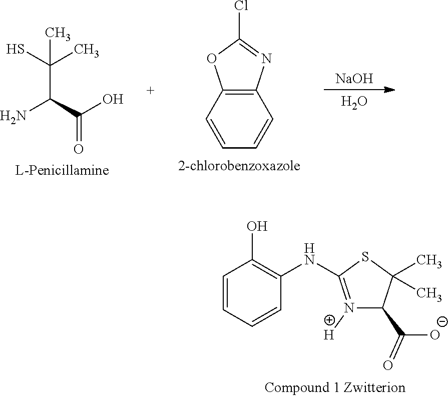

Purified water (8 volumes) was degassed with argon for approximately 30 minutes. L-penicillamine (1.6756 mol.) was added and stirred for approximately 10 minutes maintaining the temperature below 30° C. The mixture was cooled to 10±5° C. A cooled solution of sodium hydroxide (3.3512 mol.) in degassed water (2 volumes) was added slowly to the above mass while maintaining temperature below 20° C., followed by slow addition of 2-chlorobenzoxazole (1.8431 mol) below 30° C. After complete addition the reaction mass was allowed to reach ambient temperature and was stirred for not less than 8 h at ambient temperature. Upon completion of the reaction, the reaction mixture was cooled to 10±5° C., diluted with iso-propyl alcohol (10 volumes) and acidified to pH 4.3-4.6 by dropwise addition of 2N aqueous hydrochloric acid below 30° C. The solution was stirred for approximately 16 h at below 5±5° C. The solid was isolated by filtration, washed with iso-propyl alcohol (3 volumes), and dried to get the zwitterion as white solid.

### Example 2: Preparation of Compound 1 from Compound 1 Zwitterion

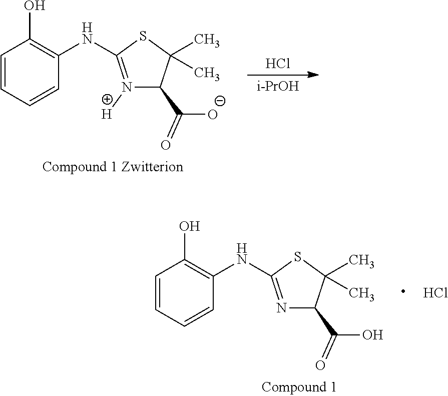

The zwitterion was added to iso-propyl alcohol (17.5 volumes) and cooled to 5±5° C. Freshly prepared 2M HCl in iso-propyl alcohol (1.05 equivalents with regard to zwitterion) was added below 10° C. The mixture was stirred for approximately 15 min, and the clear solution filtered under inert atmosphere. The filtrate was stirred not less than 16 h at 5±5° C. The mixture was concentrated to approximately 3 volumes below 30° C., methyl tert-butyl ether (MTBE) was added (5 volumes) and kept at 5±5° C. for not less than 20 h. The solid formed was isolated by filtration and washed with MTBE (3 volumes). The isolated solid was dried in vacuum tray drier at 50±5° C. for approximately 12 h to obtain Compound 1 as crystalline white solid.

### Example 3: Preparation of Des-HCl Compound 1

des-HCl Compound 1 (i.e. lacking the HCl addition salt of Compound 1) can be prepared according to Scheme 4:

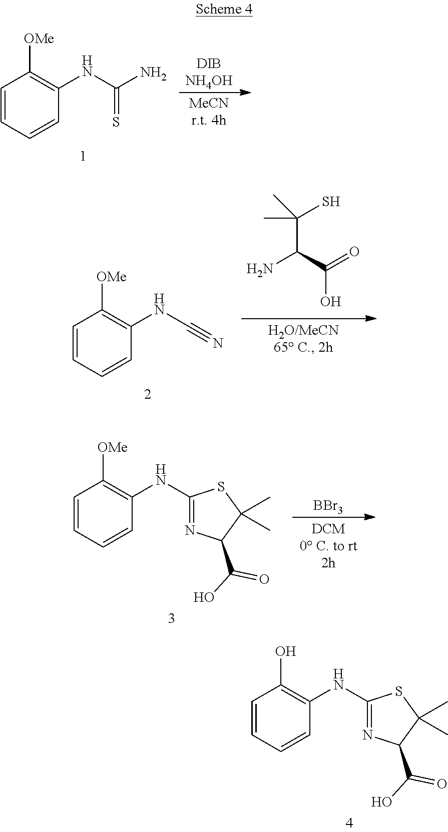

### (i) Preparation of N-(2-Methoxyphenyl)cyanamide (2)

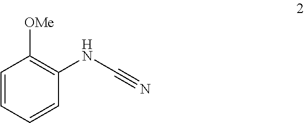 [SMILES: COc1ccccc1NC#N]

Aqueous ammonia (25%, 90 mL) was added to a stirred and ice-cooled suspension of 1-(2-methoxyphenyl)thiourea (1) (5.00 g, 27.44 mmol) in acetonitrile (90 mL). Diacetoxyiodobenzene (10.60 g, 32.92 mmol) was added portion-wise over a period of 10 min. The reaction mixture was stirred at room temperature for 4 h, and the precipitated sulfur was filtered. The filtrate was concentrated to approximately 50% of its initial volume and extracted with ethyl acetate (3×20 mL). The ethyl acetate layer was washed with water (2×30 mL) and then with brine (50 mL). The organic layer was dried over anhydrous solid Na₂SO₄, filtered and the filtrate concentrated under reduced pressure. The resultant residue was purified by flash column chromatography using petroleum ether/ethyl ether (1:1) to give the N-(2-methoxyphenyl)-cyanamide (2) (3.33 g, 82% yield). 300 MHz ¹H-NMR (CDCl₃, ppm): 7.08 (ddd, J=7.5, 1.9, 0.5 Hz, 1H) 7.04 (ddd, J=7.5, 7.5, 1.9 Hz) 6.98 (ddd, J=7.5, 7.5, 1.7 Hz) 6.88 (dd, J=7.5, 1.7 Hz) 6.26 (s, 1H) 3.88 (s, 3H). ESI-MS (m/z): 149 [M+H]⁺.

### (ii) Preparation of ((R)-2-((2-methoxyphenyl)amino)-5,5-dimethyl-4,5-dihydrothiazole-4-carboxylic Acid (3)

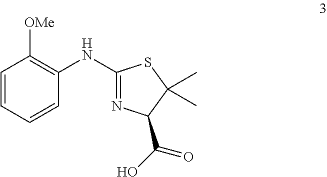 [SMILES: COc1ccccc1NC1=N[C@H](C(=O)O)C(C)(C)S1]

A mixture N-(2-methoxyphenyl)cyanamide (2) (1.00 g, 6.75 mmol) and L-penicillamine (1.21 g, 8.10 mmol) in deionized water/acetonitrile (20 mL/20 mL) was heated at reflux under an argon atmosphere for 2 h. The mixture was then concentrated under reduced pressure, and residue purified by reverse phase chromatography to afford (R)-2-((2-methoxyphenyl)amino)-5,5-dimethyl-4,5-dihydrothiazole-4-carboxylic acid (3) (0.92 g, 49% yield). 300 MHz ¹H-NMR (CD₃OD, ppm): 7.43-7.33 (m, 2H) 7.15 (dd, J=8.3, 1.1 Hz, 1H) 7.03 (ddd, J=7.7, 7.7, 1.2 Hz) 4.42 (s, 1H) 3.91 (s, 3H) 1.77 (s, 3H) 1.60 (s, 3H). ESI-MS (m/z): 281 [M+H]⁺.

### (iii) Preparation of (R)-2-((2-hydroxyphenyl)amino)-5,5-dimethyl-4,5-dihydrothiazole-4-carboxylic Acid (4)

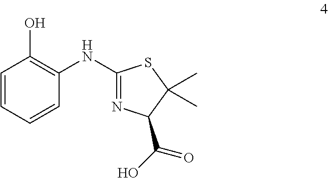 [SMILES: CC1(C)SC(Nc2ccccc2O)=N[C@@H]1C(=O)O]

Neat BBr₃ (2.19 mL, 12.84 mmol) was added to a solution of ((R)-2-((2-methoxyphenyl)amino)-5,5-dimethyl-4,5-dihydrothiazole-4-carboxylic acid (3) (360 mg, 1.28 mmol) in CH₂Cl₂ (20 mL) at 0° C. The reaction mixture was stirred at ambient temperature for 3 h, then water (2 mL) was added and the resulting suspension was stirred for 10 min. The resultant precipitate was filtered and removed. The filtrate was evaporated and purified by reverse phase chromatography to afford (R)-2-((2-hydroxyphenyl)amino)-5,5-dimethyl-4,5-dihydrothiazole-4-carboxylic acid (4) (210 mg, 64% yield). 300 MHz ¹H-NMR (CD₃OD, ppm): 6.94-6.86 (m, 2H) 6.82-6.77 (m, 1H) 6.73 (ddd, J=7.5, 7.5, 1.5 Hz) 4.19 (s, 1H) 3.91-1.68 (s, 3H) 1.49 (s, 3H). ESI-MS (m/z): 267 [M+H]⁺.

### Example 4: Amorphous Compound 1

An amorphous form of Compound 1 can also be prepared as follows:

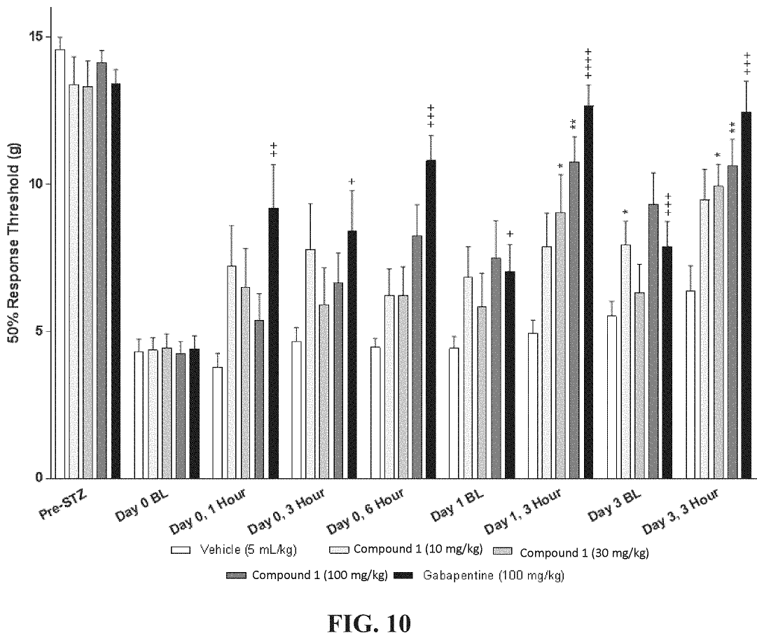

(R)-2-(2-hydroxyphenylamino)-5,5-dimethyl-4,5-dihydrothiazole-4-carboxylic acid mono-hydrochloride (Compound 1, 200 mg) was dissolved in tert-butanol:water system (1:1 ratio, 40 vol., 8 ml) at RT. The solution was filtered to remove potential seeds, and the filtered solution was frozen in a round bottom flask over a bath of dry ice and acetone. The sample was then set for freeze-drying. The XPRD of the recovered solid after freeze-drying, which is amorphous Compound 1, is shown in FIG. 10.

### Example 5: Analytical Testing of Batches of Compound 1

Starting materials for the preparation of Compound 1 are commercially available and are tested to ensure that acceptance criteria are met prior to use. The specifications for starting materials (L)-penicillamine and 2-chlorobenzoxazole are provided in Table 6.

TABLE 6
Starting Material Specifications
(L)-Penicillamine Release Specifications

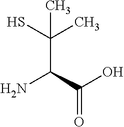

| Test Attribute | Release Specification |
|---|---|
| Appearance | Off-white to white solid |
| Identification by ¹H-NMR, IR, and Mass | Complies with the structure |
| Spectroscopy | |
| Chromatographic Purity by HPLC (ELSD) | NLT ^{a} 98.5% |
| Total Impurities | NMT ^{b} 1.5% |
| Dimer | NMT 1.0% |
| Chiral Purity by HPLC | NLT 99.0% |
| Loss on Drying | NMT 1.0% |

2-Chlorobenzoxazole Release Specifications

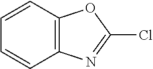

| Test Attribute | Release Specification |
|---|---|
| Appearance | Colorless to pale yellow liquid |
| Identification by 1H-NMR | Complies with structure |
| Purity (area %) by GC | NLT ^{a} 98.0% |
| BO-Imp-1 | NMT ^{b} 1.0% |

^{a} NLT = not less than
^{b} NMT = not more than

Batches of Compound 1 suitable for administration to individuals and prepared according to the method describe herein were analyzed for purity.

TABLE 7
In-Process Testing for Compound 1

| | | | |
|---|---|---|---|
| Step | Test | Method | Action Limit |

Step 1: Preparation of Compound 1 Zwitterion

| | | | |
|---|---|---|---|
| After Initial | % L-Penicillamine | HPLC ELSD | NMT ^{a} 1.0% |
| Reaction | | | |
| After Initial | Water Content | Karl Fischer | NMT 1.0% |
| Drying | Purity and | HPLC UV | Zwitterion: NLT ^{b} |
| | Related | | 98.5% 2-Cl |
| | Substances of | | BO: NMT |
| | Zwitterion | | 0.15% BO-Imp- |
| | | | 1: NMT 0.15% |
| | | | BO-Imp-2: NMT 0.15% |
| | | | BO-Imp-3: NMT 0.15% |
| | | | BO-Imp-4: NMT 0.15% |
| | | | BO-Imp-5: NMT 0.15% |
| | | | Cmpd 1-Imp-3: NMT |
| | | | 0.5% |
| | % L-Penicillamine | HPLC ELSD | NMT 0.3% |
| | Chiral Impurity | HPLC UV | NMT 1.0% |
| | Benzene | GC | NMT 2 ppm |
| | Triethylamine | GC-MS | NMT 320 ppm |
| After Final Drying | Loss on Drying | USP <731> | Report result |
| | Residue on Ignition | USP <281> | Report result |

Step 2: Preparation of Compound 1

| | | | |
|---|---|---|---|
| Iso-propyl | Molarity | Titration | Report result |
| Alcohol/HCl | | | |
| After Initial | Purity and | HPLC UV | Purity: NLT 98.5% |
| Reaction | Related | | 2-Cl BO: NMT |
| | Substances | | 0.15% BO-Imp- |
| | | | 1: NMT 0.15% |
| | | | BO-Imp-2: NMT 0.15% |
| | | | BO-Imp-3: NMT 0.15% |
| | | | BO-Imp-4: NMT 0.15% |
| | | | BO-Imp-5: NMT 0.15% |
| | | | Compound 1-Imp-3: |
| | | | NMT 0.5% |
| | | | Unspecified |
| | | | Impurities: |
| | | | NMT 0.15% |
| | Chiral Impurity | HPLC UV | NMT 1.0% |
| | % L-Penicillamine | HPLC ELSD | NMT 0.5% |
| | Residue on Ignition | ROI | Report results |
| After Purification | Residue on Ignition | ROI | NMT 0.25% |
| After Drying | Residual Solvents | GC | Ethanol: NMT 5,000 |
| | | | ppm; |
| | | | n-Butanol: NMT |
| | | | 5,000 ppm; |
| | | | Iso-propyl alcohol: |
| | | | NMT 5,000 ppm; |
| | | | Methyl tert- |
| | | | butyl ether: |
| | | | NMT 5,000 |
| | | | ppm; |
| | | | Chloroform: NMT |
| | | | 60 ppm ; 1,2- |
| | | | Dichloroethane: |
| | | | NMT 5 ppm |
| | Water | Karl Fischer | NMT 1.0% |

^{a} NMT = not more than
^{b} NLT = not less than

### Example 6: Analytical Methods Used in Testing Compound 1

Analytical methods, in various embodiments, were carried out with equipment and parameters set forth below. The testing was conducted on batches Compound 1 suitable for administration to individuals according to the methods and specifications belonging to the USP (United States Pharmacopeia).

TABLE 8
Analytical Procedures For Compound 1

| Test | Summary of the Analytical Procedure | | | |
|---|---|---|---|---|
| Description | Visual Examination | | | |
| IR Identification | FT-IR | | | |
| HPLC Method 1 | Identification is confirmed by verifying the retention time of the Compound 1 | | | |
| Identification, | peak in the drug substance is consistent with that of the working standard. | | | |
| Purity, Assay, | Purity, assay, and related substances are performed using reversed-phase | | | |
| and Impurities | HPLC and the following chromatographic conditions. | | | |
| BO-Imp⁻1, | Instrument | Suitable HPLC with variable wavelength UV | | |
| BO-Imp-4, | | detector | | |
| BO-Imp-5, | Column | X-Bridge C18, 250 × 4.6 mm, 5 μm | | |
| Compound 1 Imp-3, | Mobile Phase A | 25 mM K₂HPO₄ in water, pH 8.4:Methanol (95:5) | | |
| Individual | Mobile Phase B | Acetonitrile:Methanol (50:50) | | |
| Unspecified | | | % Mobile | % |
| Impurities, | Gradient | Time | Phase A | Mobile |
| Total Impurities | | 0.01 | 75 | 25 |
| | | 2.00 | 75 | 25 |
| | | 12.00 | 55 | 45 |
| | | 18.00 | 55 | 45 |
| | | 35.00 | 35 | 65 |
| | | 40.00 | 35 | 65 |
| | | 40.10 | 75 | 25 |
| | | 50.00 | 75 | 25 |
| | Flow Rate | 1.0 mL/min | | |
| | Injection Volume | 8.0 μL | | |
| | Wavelength | 225 nm | | |
| | Column | 30° C. | | |
| | Temperatur | | | |
| | Detector | 40° C. | | |
| | Cell | | | |
| | Run Time | 50 minutes | | |
| HPLC Method 2 | A limit test is performed for process impurities 2-Cl-BO and BO-Imp-2 are | | | |
| Limit Test 2-Cl- | performed using reversed-phase HPLC and the following chromatographic | | | |
| BO and BO-Imp-2 | conditions. | | | |
| | Instrument | Suitable HPLC with variable wavelength UV | | |
| | | detector | | |
| | Column | X-Bridge C18, 250 × 4.6 mm, 5 μm | | |
| | Mobile Phase A | 25 mM K₂HPO₄ in water, pH 8.4:Methanol (95:5) | | |
| | Mobile Phase B | Acetonitrile:Methanol (50:50) | | |
| | | | % Mobile | % |
| | Gradient | Time | Phase A | Mobile |
| | | 0.01 | 75 | 25 |
| | | 2 | 75 | 25 |
| | | 12 | 55 | 45 |
| | | 18 | 55 | 45 |
| | | 35 | 35 | 65 |
| | | 40 | 35 | 65 |
| | | 40.1 | 75 | 25 |
| | | 50 | 75 | 25 |
| | Flow Rate | 1.0 mL/min | | |
| | Injection Volume | 10.0 μL | | |
| | Wavelength | 250 nm | | |
| | Column | 30° C. | | |
| | Temperatur | | | |
| | Detector | 40° C. | | |
| | Cell | | | |
| | Run Time | 50 minutes | | |
| HPLC Method | A limit test for BO-Imp-3 is performed using reversed-phase HPLC and the | | | |
| 3 B0-Imp-3 | following chromatographic conditions. | | | |
| | Instrument | Suitable HPLC with variable wavelength UV | | |
| | | detector | | |
| | Column | Waters X-Bridge C18, 250 × 4.6 mm, 5 μm | | |
| | Mobile Phase A | 25 mM K₂HPO₄ in water, pH 8.4:methanol (95:5) | | |
| | Mobile Phase B | Acetonitrile:Methanol (50:50) | | |
| | | | % | % |
| | Gradient | Time | Mobile | Mobile |
| | | 0.01 | 75 | 25 |
| | | 2 | 75 | 25 |
| | | 12 | 55 | 45 |
| | | 18 | 55 | 45 |
| | | 35 | 35 | 65 |
| | | 40 | 35 | 65 |
| | | 40.1 | 75 | 25 |
| | | 50 | 75 | 25 |
| | Flow Rate | 1.0 mL/min | | |
| | Injection Volume | 10 μL | | |
| | Wavelength | 225 nm | | |
| | Column | 30° C. | | |
| | Temperatur | | | |
| | Autosampler | 15° C. | | |
| | Detector | 40° C. | | |
| | Cell | | | |
| | Run Time | 50 minutes | | |
| HPLC Method 4 | A limit test for L-penicillamine is performed using reversed-phase HPLC | | | |
| (L)-Penicillamine | using a MS detector and the following chromatographic conditions. | | | |
| | | | % | % |
| | Gradient | Time | Mobile | Mobile |
| | | 0.0 | 100 | 0 |
| | | 10 | 20 | 80 |
| | | 16 | 20 | 80 |
| | | 17 | 100 | 0 |
| | | 22 | 100 | 0 |
| | Flow Rate | 0.5 mL/min | | |
| | Injection Volume | 10 μL | | |
| | Wavelength | 254 nm | | |
| | Column | 35° C. | | |
| | Temperature | | | |
| | Run Time | 22 minutes | | |
| | Mass Parameters | | | |
| | Nebulizer Pressure | 40 psi | | |
| | Dry Gas Flow Rate | 10 L/min | | |
| | Fragmentor Voltage | 70 V | | |
| | Capillary Voltage | 3,000 V | | |
| | Dry Gas | 350° C. | | |
| | Temperature | | | |
| | Collection Mode | SIM mode: positive signal for 150 ion | | |
| HPLC Method 5 | A limit test for BO-Imp-3 is performed using chiral HPLC and the following | | | |
| S-Compound 1 Imp- | chromatographic conditions. | | | |
| 3 | Instrument | Suitable HPLC with variable wavelength UV | | |
| | | detector | | |
| | Column | Chiralpak IG, 250 × 4.6 mm, 5 μm | | |
| | Mobile Phase: | 0.1% diethylamine in acetonitrile:methanol 95:5 | | |
| | Flow Rate | 0.8 mL/min | | |
| | Injection Volume | 10 μL | | |
| | Wavelength | 285 nm | | |
| | Column | 25° C. | | |
| | Temperature | | | |
| | Autosampler | 25° C. | | |
| | Temperature | | | |
| | Detector Cell | 40° C. | | |
| | Temperature | | | |
| | Run Time | 70 minutes | | |
| HPLC Method | Quantitation of (S)-Compound 1 is performed using chiral HPLC | | | |
| 6 Chiral Purity | chromatography and the following chromatographic conditions. | | | |
| | Instrument | Suitable HPLC with variable wavelength detector | | |
| | Column | Chiralcel OX-3, 250 × 4.6 mm, 3 μm | | |
| | Mobile Phase A | 0.3% trifluoroacetic acid in n-hexane | | |
| | Mobile Phase B | 0.1% diethylamine in ethanol:iso-propyl alcohol 8:2 | | |
| | | | % Mobile | % Mobile |
| | Gradient | Time | Phase A | Phase B |
| | | 0.01 | 80 | 20 |
| | | 15.0 | 80 | 20 |
| | Flow Rate | 1.0 mL/min | | |
| | Injection Volume | 10 μL | | |
| | Wavelength | 285 nm | | |
| | Column | 25° C. | | |
| | Temperature | | | |
| | Detector Cell | 40° C. | | |
| | Temperature | | | |
| | Run Time | 15 minutes | | |
| Residual Solvents | Quantitation of ethanol, iso-propyl alcohol, n-butanol, and methyl tert- | | | |
| Ethanol, Iso-propyl | butyl ether is performed using a headspace GC method and flame | | | |
| Alcohol, n- | ionization detection. The chromatographic conditions are listed below. | | | |
| Butanol, MTBE ^{a} | Instrument | Suitable GC with flame ionization detector (FID) | | |
| | Column | DB-1, 60 m × 0.32 mm, 3 μm | | |
| | Carrier Gas | Helium | | |
| | Temperature | Rate | Temperature | Hold Time |
| | Program | (° C./min) | (° C.) | (Minutes) |
| | | — | 50 | 2 |
| | | 3 | 80 | 5 |
| | | 15 | 260 | 11 |

| | Flow Rate | 1.5 mL/min |
|---|---|---|
| | Injection Mode | Split |
| | Split Ratio | 10:1 |
| | Detector | 280° C. |
| | Temperature | |
| | Make-Up Gas | Helium |
| | Make-Up Flow | 30.0 mL/min |
| | H₂ Flow | 40.0 mL/min |
| | Air Flow | 400.0 mL/min |
| | Run Time | 40.0 minutes |
| Residual | Quantitation of chloroform is performed using a GC method and electron | |
| Solvent | impact mass detection. The chromatographic conditions are listed below. | |
| Chloroform | Instrument | Suitable GC with electron impact mass detection |
| | Column | DB-1, 60 m × 0.32 mm, 3 μm |
| | Carrier Gas | Helium |
| | Oven Temperature | 50° C., hold at ° C. for 2 minutes |
| | Temperature Ramp | 50° C. to 80° C. at 3° C./min, hold at 80° C. for 7 minutes |
| | | 80° C. to 260° C. at 50° C./min, hold at 260° C. for |
| | | 12 minutes |
| | Flow Rate | 1.0 mL/min |
| | Injection Mode | Split |
| | Split Ratio | 10:1 |
| | Injector | 200° C. |
| | Temperature | |
| | Injection Volume | 2 μL |
| | Make-Up Flow | 30.0 mL/min |
| | Run Time | 34.6 minutes |
| Residual Solvent | Quantitation of 1,2-Dichloroethane is performed using a GC method and | |
| 1,2- | electron impact mass detection. The chromatographic conditions are listed | |
| Dichloroethane | below. | |
| | Instrument | Suitable GC with electron impact mass detection |
| | Column | DB-624, 30 m × 0.32 mm, 1.8 μm |
| | Carrier Gas | Helium |
| | Oven Temperature | 40° C., hold at 40° C. for 5 minutes |
| | Temperature Ramp | 40° C. to 60° C. at 4° C./min, hold at 60° C. for 1 minute |
| | | 60° C. to 250° C. at 50° C./min, hold at 250° C. for |
| | | 6 minutes |
| | Flow Rate | 1.5 mL/min |
| | Injection Mode | Split |
| | Split Ratio | 5:1 |
| | Injector Temperature | 220° C. |
| | Injection Volume | 1 μL |
| | Run Time | 20.8 minutes |

| | |
|---|---|
| Water | USP <921>, Method Ia |
| Residue on Ignition | USP <281> |
| Elemental | Arsenic (As), cadmium (Cd), mercury (Hg), lead (Pb), cobalt (Co), |
| Impurities Arsenic, | vanadium (V), and nickel (Ni) content are determined using Inductively |
| Cadmium, | Coupled Plasma (ICP) with mass spectral detection. |
| Mercury, Lead, | |
| Cobalt, Vanadium, | |
| and Nickel | |
| Elemental | Lithium (Li), antimony (Sb), and copper (Cu), content are determined |
| Impurities | using ICP with Optical Emission Spectroscopy (OES) detection. |
| Lithium, | |
| Powder XRD | USP <941> |
| Microbial Analysis | USP <61>, USP <62> |

In various embodiments, the methods described herein produce Compound 1 with one or more of the parameters, such amounts of impurities, set forth in Table 9:

TABLE 9
Compound 1 Specifications

| Parameter | Test Method | Specification (Acceptance Criteria Applied) |
|---|---|---|
| Description | Visual | White to off-white solid |
| | Examination | |
| Identification | | |
| IR | FT-IR | Conforms to structure |
| HPLC | HPLC Method 1 | The retention time of the principal peak in the sample |
| | | chromatogram corresponds to that of the standard |
| | | chromatogram |
| Chloride | USP <191> | With Silver Nitrate TS, solution of chlorides yield a white, |
| | Test A | curdy precipitate that is insoluble in nitric acid but is |
| | | soluble in a slight excess of 6N ammonium hydroxide |
| Purity | HPLC Method 1 | NLT^{a} 98.5% (% area) |
| Assay | HPLC Method 1 | 97.0%-103.0% |
| Impurities | | |
| 2-Cl-BO | HPLC Method 2 | NMT 0.004% |
| BO-Imp-1 | HPLC Method 1 | NMT 0.15% |
| BO-Imp-2 | HPLC Method 2 | NMT 0.004% |
| BO-Imp-3 (2-aminophenol) | HPLC Method 3 | NMT 0.004% |
| BO-Imp-4 | HPLC Method 1 | NMT 0.15% |
| BO-Imp-5 | | NMT 0.15% |
| Compound 1 Imp-3 | | NMT 0.5% |
| L-Penicillamine | HPLC Method 4 | NMT 0.004% |
| S-Compound 1 Imp-3 | HPLC Method 5 | NMT 0.15% |
| Chiral Purity | HPLC Method 6 | NMTb 0.5% S-Isomer |
| Any Individual Unspecified | HPLC Method 1 | NMT 0.15% |
| Impurity | | |
| Total Impurities | | NMT 1.5% |
| Residual Solvents | | |
| Ethanol | GC-HS Method 1 | NMT 5,000 ppm |
| Iso-propyl Alcohol | | NMT 5,000 ppm |
| n-Butanol | | NMT 5,000 ppm |
| Methyl tert-butyl Ether | | NMT 5,000 ppm |
| Chloroform | GC-MS Method 2 | NMT 60 ppm |
| 1,2-Dichloroethane | GC-MS Method 3 | NMT 5 ppm |
| Water | Karl Fischer | NMT 1.0% (w/w) |
| Residue on Ignition | USP <281> | NMT 0.25% w/w |
| Elemental Impurities | | |
| Arsenic | ICP-MS | NMT 1.5 ppm |
| Cadmium | | NMT 0.2 ppm |
| Mercury | | NMT 0.3 ppm |
| Lead | | NMT 0.5 ppm |
| Cobalt | | NMT 0.5 ppm |
| Vanadium | | NMT 1 ppm |
| Nickel | | NMT 2 ppm |
| Lithium | ICP-OES | NMT 55 ppm |
| Antimony | | NMT 120 ppm |
| Copper | | NMT 300 ppm |
| Powder XRD | XRPD | Crystalline |
| Microbial Analysis | | |
| TAMC | USP <61>, | NMT 10³ in 1 g |
| | USP <62> | |
| TYMC | | NMT 10² in 1 g |
| E. coli | | Absent in 1 g |

In various embodiments, Compound 1 produced according to the methods described herein has one or more of the analytical parameters, including amounts of impurities, set forth in Table 10.

TABLE 10
Data for Compound 1 Drug Substance Batches

| Batch Number | | |
|---|---|---|
| Attribute | Proposed Specifications | Drug Batch |
| Description | White to off-white solid | White solid |
| Identification | | |
| ¹H-NMR ^{a} | Conforms to structure | NT |
| LC-MS ^{a} | Conforms to m/z | NT |
| IR | IR spectrum conforms to the | Complies |
| | structure of the molecule | |
| HPLC | The retention time of the principal | Complies |
| | peak in the sample chromatogram | |
| | corresponds to that of the standard | |
| | chromatogram | |
| Chloride | With Silver Nitrate TS, solution of | Complies |
| | chlorides yields a white, curdy | |
| | precipitate that is insoluble in nitric | |
| | acid but is soluble in a slight excess | |
| | of 6N ammonium hydroxide | |
| Purity | NLT 98.5% (% area) | 99.7 |
| Assay | 97.0% 0 103.0% | 100.8 |
| Specified Impurities | | |
| 2-Cl-BO | NMT 0.004% | <0.004 ^{d} |
| BO-Imp-1 | NMT 0.15% | 0.05 |
| BO-Imp-2 | NMT 0.004% | <0.004 ^{d} |
| BO-Imp-3 (2-aminophenol) | NMT 0.004% | <0.004 ^{d} |
| BO-Imp-4 | NMT 0.15% | <0.013 (LOD ^{e}) |
| BO-Imp-5 | NMT 0.15% | <0.045 (LOQ ^{f}) |
| Compound 1 Imp-3 | NMT 0.5% | 0.16 |

TABLE 11
Release Data for Compound 1 Drug Substance Batches

| Batch Number | | | |
|---|---|---|---|
| Attribute | Proposed Specifications | Drug Batch | |
| L-Penicillamine | NMT 0.004% | <0.004 ^{d} | |
| S-Compound 1 Imp-3 | NMT 0.15% | <0.15 ^{d} | |
| Chiral Purity (S- | NMT 0.5% | <0.030 | (LOD) |
| Any Individual | NMT 0.15% | | |
| Unspecified | RRT 1.54 | <0.049 | (LOQ) |
| Impurity | RRT 1.85 | 0.11 | |
| | RRT 2.49 | <0.049 | (LOQ) |
| | RRT 3.27 | ND | |
| | RRT 3.87 | <0.049 | (LOQ) |
| | RRT 3.95 | <0.049 | (LOQ) |
| Total Impurities | NMT 1.5% | 0.3 | |
| Residual Solvents | | | |
| Ethanol | NMT 5,000 ppm | <150 ppm | (LOD) |
| Iso-propyl Alcohol | NMT 5,000 ppm | 3,507 ppm | |
| n-Butanol | NMT 5,000 ppm | <150 ppm | (LOD) |
| MTBE | NMT 5,000 ppm | <150 ppm | (LOD) |
| Chloroform | NMT 60 ppm | <3.6 ppm | (LOD) |
| 1,2-Dichloroethane | NMT 5 ppm | <0.4 ppm | (LOD) |
| Water | NMT 1.0% (w/w) | 0.18 | |
| Residue on Ignition | NMT 0.25% w/w | 0.06 | |
| Elemental Impurities | | | |
| Arsenic | NMT 1.5 ppm | <0.225 ppm | (PDL) ^{g} |
| Cadmium | NMT 0.2 ppm | <0.03 ppm | (PDL) |
| Mercury | NMT 0.3 ppm | <0.045 ppm | (PDL) |
| Lead | NMT 0.5 ppm | <0.075 ppm | (PDL) |
| Cobalt | NMT 0.5 ppm | <0.15 ppm | (PDL) |
| Vanadium | NMT 1 ppm | <0.075 ppm | (PDL) |

| | | |
|---|---|---|
| Nickel | NMT 2 ppm | <1.51 ppm |
| Lithium | NMT 55 ppm | <3 ppm |
| Antimony | NMT 120 ppm | <3 ppm |
| Copper | NMT 300 ppm | <3 ppm |
| Powder XRD | Crystalline | Crystalline |
| Microbial Analysis | | |
| TAMC | NMT 10³ cfu in 1 g | <10 |
| TYMC | NMT 10² cfu in 1 g | <10 |
| E. coli | Absent in 1 g | Absent |

^{a} Testing performed for Batch A011800996 and is not required for routine release.
^{b} NT = not tested
^{c} ND = not detected
^{d} Result obtained after development and qualification of Methods 2-5.
^{e} LOD = limit of detection
^{f} LOQ = limit of quantitation
^{g} PDL = practical detection limit

### Example 7: Pharmacology Overview

Compound 1 is a non-metal, orally bioavailable small molecule Reactive Species Decomposition Accelerant (RSDAx) which, in various embodiments, destroys peroxynitrite (PN) and/or hydrogen peroxide. Peroxynitrite and peroxide are powerful oxidants produced under conditions of injury and disease that cause untoward effects via protein nitration and modification of sensory ion channels leading to neuronal sensitization and pain.

In chemical-based assays of peroxynitrite (PN) oxidation, Compound 1 inhibits PN-mediated oxidation of small-molecule organic substrates such as luminol. In cell-based assays of PN-mediated cytotoxicity, Compound 1 is protective. Compound 1 can also catalytically remove peroxynitrite in models of protein nitration (a consequence of peroxynitrite oxidation) and in lactoperoxidase oxidation (mediated by peroxide) under physiological conditions (i.e., neutral pH). Chemically, Compound 1 can also react stoichiometrically with peroxynitrite to form a para-nitro adduct. Without being bound by theory, by targeting and removing peroxynitrite and peroxide, Compound 1 can disrupt the ensuing cascades that lead to hypersensitivity (protein modification, ion channel hyperexcitation) thus providing a long duration event in terms of pain relief.

In various embodiments, Compound 1 alleviates allodynia in rat models of diabetic neuropathy (streptozotocin- and methylglyoxal-induced) without brain penetration, thereby avoiding common CNS side effects associated with gabapentin and duloxetine. In various embodiments, Compound 1 does not penetrate the blood-brain barrier (BBB). In various embodiments, less than about 3%, 2%, 1%, 0.8%, 0.6%, 0.4%, 0.2%, 0.1%, 0.08%, 0.06%, 0.04%, 0.02%, or 0.01% of Compound 1 in blood plasma penetrates the BBB. Compound 1 does not alter normal sensation when given to uninjured animals.

Compound 1 rapidly produces complete reversal of hypersensitivity caused by an injury/insult such as an incision or irritant and upon repeated dosing, reverses allodynia in models of painful diabetic neuropathy. Compound 1 was examined in a variety of pharmacokinetic and metabolism studies. The compound was examined in detail in rat and dog, the species selected for toxicology studies. In vivo, no epimerization of Compound 1 was found using chiral methods. The compound is bioavailable after oral administration in both rat and dog.

Upon administration, Compound 1 is stable in both plasma and hepatocytes from rat, dog and human. Compound 1 is excreted into urine and feces of rats primarily as a sulfate conjugate. Compound 1 distributes to tissues but not to brain to an appreciable extent. Compound 1 is moderately protein-bound across species. Compound 1 does not inhibit major CYP isoforms (IC₅₀ for CYPs 3A4, 2D6, 1A2, 2C9, 2C19 are all >100 μM). Compound 1 does not inhibit P-gp, OATP1B1, OATP1B3 and OAT1, weakly inhibits OAT3 and modestly inhibits BCRP, which suggests that interactions with transporters or inhibition of CYPs would be minimal or absent at pharmacologically active doses.

### Example 8: Compound 1 Effect on Allodynia in Rodent Models of Diabetic Neuropathy

Experiments were conducted to determine the effect of Compound 1 in streptozotocin (STZ)-induced diabetic rats. In the first experiment, after obtaining mechanical threshold baselines, STZ (50 mg/kg IV) was administered (Day −7). Two days later, blood glucose levels were measured and animals that were hyperglycemic (>250 mg/dL) continued in the study. On Day 0 the baseline for mechanical thresholds prior to dosing with test articles was established for the rats. Compound 1 (10, 30, and 100 mg/kg PO), gabapentin (100 mg/kg PO) or vehicle was then administered daily for 5 days. On Day 0 mechanical thresholds were obtained at 1, 3, and 6 h post-dose. On Day 1 mechanical thresholds were measured immediately prior to dosing and then 3 h post-dose. The same regimen was followed on Day 3. The gabapentin cohort exhibited a significant reversal of allodynia 1 h post-dose on Day 0 and all time points thereafter. Compound 1-treated groups showed non-significant reversal of allodynia on Day 0 which became statistically-significant on subsequent days, evident by increases in mechanical threshold values relative to vehicle, and these effects were comparable to gabapentin (FIG. 14).

In a subsequent study, the same STZ paradigm was followed. However, in addition to the 100 mg/kg Compound 1 group, two additional cohorts received the Compound 1 100 mg/kg dose split into two or three doses per day with 50 mg/kg BID and 33 mg/kg TID. In addition to the gabapentin (100 mg/kg PO) group, a separate cohort received duloxetine (30 mg/kg PO). Additionally, in this study mechanical threshold testing was implemented every day immediately prior to dosing and 2 h post-dose for 7 days. Compound 1 reversed allodynia upon continued daily dosing reaching statistical significance from Day 1 and beyond. There was no clear difference between Compound 1-treated groups regarding dosing regimen (100 mg/kg QD vs 50 mg/kg BID vs 33 mg/kg TID). Gabapentin robustly reversed the allodynia throughout the study whereas the duloxetine cohort required successive doses for appreciable activity.

### Example 9: Methods of Analysis for Pharmacokinetics Measurements

Formulation analysis and bioanalytical methods were validated for all GLP studies. Formulation of Compound 1 in water was validated over a range of 1 to 200 mg/mL using HPLC and in 0.5% hydroxypropyl methylcellulose (HPMC) over the same range.

Compound 1 concentration in rat plasma was validated using LC/MS/MS with a lower limit of quantitation of 0.1 μg/mL using a 50 μL sample. Similar conditions were used to validate a bioanalytical method in dog plasma also using 50 μL of plasma. In both assays, a deuterated (Compound 1-d₄) internal standard was used.

Dose formulation analysis for all good laboratory practice (GLP) general toxicology studies was performed using a validated high-performance liquid chromatography (HPLC)/ultraviolet (UV) analytical method (2750-001-001 Dose Formulation Method 1). The vehicle used in the in vivo toxicology studies was 0.5% HPMC). The analytical method utilized HPLC with monitoring at 227 nm with an isocratic mobile phase of methanol with the column temperature set to 25° C. Linearity over a range of 1.0 to 200 mg/mL was observed. Dose formulations over this range were stable at room temperature for up to 13 days and were stable when stored frozen at −20° C. for up to 85 days.

In addition, formulation analysis for in vitro genetic toxicology studies was performed using a validated HPLC/UV analytical method (2750-001-001 Dose Formulation Method No. 2) with linearity over a range of 0.001 to 50 mg/mL. Dose formulations for in vitro assays were stable at room temperature for up to 1 day and when stored frozen at −20° C. for up to 45 days.

### Example 10: Distribution of Compound 1

A rat brain distribution study performed in order to determine standard pharmacokinetic parameters and to assess the brain penetration properties of Compound 1 administered orally and intravenously. The compound was rapidly and completely absorbed orally in rats after administration of a 30 mg/kg dose, reaching a peak plasma concentration of 9 μg/mL within 1 hr.

The compound distributed readily in tissues with a steady-state volume of distribution of 1.7 L/kg. Compound 1 was peripherally restricted with a brain-to-plasma concentration ratio of 0.02 1 h after IV administration eliminated at a moderate rate from the systemic circulation. Oral bioavailability of 111% was calculated for the 30 mg/kg oral dose with a terminal half-life of 1.6 hours. A Red Blood Cell (RBC) partitioning showed that Compound 1 poorly partitions into RBC at 60 min post-exposure (K_{RBC/PL}<0.25) across all species (rat, dog, monkey, human).

### Example 11: CYP Inhibition

At varying concentrations, Compound 1 was incubated with human liver microsomes (HLM) in the presence of known substrates (see below) of specific CYP isoforms, in order to measure inhibition induced by Compound 1. Microsomes were incubated with known inhibitors (positive controls) of each CYP isoform, in the presence of substrate, in order to measure the metabolic activity of the microsomes.

TABLE 12
CYP Inhibition

| Test compound | Compound 1 |
|---|---|
| Inhibitor | CYP1A2: α-Naphthoflavone |
| (Positive control) | CYP2C9: Sulfaphenazole |
| | CYP2C19: Omeprazole |
| | CYP2D6: Quinidine |
| | CYP3A4: Ketoconazole |
| Final highest | 100 μM test compound, 20 μM α-Naphthoflavone; 10 μM |
| concentration | Sulfaphenazole; 100 μM Omeprazole; 2.5 μM Quinidine; 2.5 μM |
| | Ketoconazole |
| Substrate | CYP1A2: Phenacetin |
| | CYP2C9: Tolbutamide |
| | CYP2C19: S-Mephenytoin |
| | CYP2D6: Dextromethorphan |
| | CYP3A4: Midazolam |
| Test concentration | 30 μM Phenacetin; 100 μM Tolbutamide; 35 μM |
| | S-Mephenytoin; 5 μM Dextromethorphan; 5 μM Midazolam |
| Test systems | Human liver microsomes from BD Gentest (0.5 mg/mL for |
| | CYP1A2, 2C9, 2C19; 0.2 mg/mL for 2D6; 0.1 mg/mL for |
| | CYP3A4) |
| Incubation condition | 37° C. incubation for 10 minutes for CYP1A2; 15 minutes |
| | for CYP2C9, 2D6; 45 minutes for CYP2C19; 5 minutes for |
| | CYP3A4 |
| Sample size | Duplicate (n = 2) |
| Bioanalytical method | HPLC-MS/MS |

Compound 1 did not inhibit the five CYP isoforms tested (see table below). The positive controls produced CYP inhibition consistent with historical (and literature) values indicating that the microsomes were metabolically active and of high integrity.

| CYP isoform | IC₅₀ (μM) |
|---|---|
| CYP 3A4 | >100 |
| CYP 1A2 | >100 |
| CYP 2D6 | >100 |
| CYP 2C9 | >100 |
| CYP 2C19 | >100 |

### Example 12: Inhibition of Transporters

Compound 1 was evaluated to determine inhibition of human ATP binding cassette (ABC) transporters (known as efflux transporters) and solute-linked carrier (SLC) transporters (known as uptake transporters as outlined below:

TABLE 13
Inhibition of Transporters

| Transporter | | | |
|---|---|---|---|
| (Gene symbol) | Test system | Probe substrate | Experimental design |
| P-gp | Caco-2 cells | Digoxin | Bidirectional transport of the probe substrate |
| (MDR1/ABCB1) | | | across Caco-2 cells, MDCKII-BCRP cells |
| BCRP | MDCKII cells | Prazosin | and MDCKII control cells |
| (ABCG2) | | | |
| OATP1B1 | HEK293 cells | [³H]-Estradiol-17β- | Accumulation of the probe substrate into |
| (OATP2/OATP- | | glucuronide | transporter-expressing and control cells |
| C/SLCO1B1) | | | |
| OATP1B3 | | | |
| (OATP8/SLCO1B3) | | | |
| OAT1 | | [³H]-p-Aminohippurate | |
| (SLC22A6) | | | |
| OAT3 | | [³H]-Estrone-3-sulfate | |
| (SLC22A8) | | | |
| OCT1 | | [¹⁴C]- | |
| (SLC22A1) | | Tetraethylammonim | |
| MATE1 | | | |
| (SLC47A1) | | | |
| MATE2-K | | [¹⁴C]-Metformin | |
| (SLC47A2) | | | |

TABLE 14
Experimental Design for the In Vitro Evaluation
of Compound 1 for Inhibition of P-gp and BCRP

| | Caco-2 | MDCKII-BCRP |
|---|---|---|
| Test article | Compound 1 | Compound 1 |
| [Test article] (μM) | 1, 3, 10, 30, 100, 600 | 0.1, 0.3, 1,3, 10,30 |
| Probe substrate | Digoxin (10 μM) | Prazosin (1 μM) |
| Positive control inhibitor(s) | Valspodar (1 μM) | Ko143 (1 μM) |
| | Verapamil (60 μM) | Lopinavir (30 μM) |
| Permeability control | Lucifer yellow (40 μg/mL) | Not applicable |
| Nominal cell number per well | 0.3 × 10⁵ | 0.3 to 0.4 × 10⁶ |
| Volume per well (μL) | Apical: 200; Basal: 980 | Apical: 200; Basal: 980 |
| Preincubation time (min) | 30 to 60 | 30 to 60 |
| Incubation time (min) | Donor: 0, 120; Receiver: 120 | Donor: 0, 120; Receiver: 120 |
| Incubation temperature (° C.) | 37 ± 2 | 37 ± 2 |
| Incubation medium | HBSS supplemented with | HBSS supplemented with |
| | HEPES (25 mM) and glucose | HEPES (25 mM) and glucose |
| | (25 mM) | (25 mM) |
| Number of replicates | 3 | 3 |
| Probe substrate analysis method | LC-MS/MS | LC-MS/MS |

TABLE 15
Experimental Design for the In Vitro Evaluation of Compound
1 for the Inhibition of OATP, OAT, OCT and MATE inhibition

| | OATP1B1/ | | | | MATE1/ |
|---|---|---|---|---|---|
| | OATP1B3 | OAT1 | OAT3 | OCT1 | MATE2-K |
| Test article | Compound 1 | Compound 1 | Compound 1 | Compound 1 | Compound 1 |
| [Test article] | 0.3, 1, 3, 10, 30, | 0.3, 1, 3, 10, 30, | 0.3, 1, 3, 10, 30, | 0.3, 1, 3, 10, 30, | 0.3, 1, 3, 10, 30, |
| (μM) | 100, 600 | 100, 600 | 100, 600 | 100, 600 | 100, 600 |
| Probe | [³H]-Estradiol- | [³H]-p- | [³H]-Estrone-3- | [¹⁴C]-Tetraethyl- | [¹⁴C]-Metformin |
| substrate | 17β-glucuronide | Aminohippurate | sulfate | ammonium bromide | (10 μM) |
| | (0.05 μM) | (1 μM) | (0.05 μM) | (5 μM) | |
| Positive | Rifampin | Probenecid | Probenecid | Quinidine | Pyrimethamine |
| control | (10 μM) | (100 μM) | (100 μM) | (100 μM) | (0.1 μM for |
| inhibitor(s) | | | | | MATE1 and |
| | | | | | 0.3 μM for |
| | | | | | MATE2-K) |
| | Cyclosporine | Novobiocin | Ibuprofen | Verapamil | Cimetidine |
| | (1 μM) | (300 μM) | (100 μM) | (10 μM) | (10 μM for |
| | | | | | MATE1 and |
| | | | | | 300 μM for |
| | | | | | MATE2-K)) |
| Nominal cell | 0.2 to | 0.2 to | 0.2 to | 0.2 to | 0.2 to |
| number per well | 0.4 × 10⁶ | 0.4 × 10⁶ | 0.4 × 10⁶ | 0.4 × 10⁶ | 0.4 × 10⁶ |
| Volume per | 300 | 300 | 300 | 300 | 300 |
| well (μL) | | | | | |
| Pre-incubation | 30 | 15 | 15 | 15 | 15 |
| time (min) | | | | | |
| Incubation | 2 | 1 | 2 | 15 | 5 |
| time (min) | | | | | |
| Incubation | 37 ± 2 | 37 ± 2 | 37 ± 2 | 37 ± 2 | 37 ± 2 |
| temperature | | | | | |
| (° C.) | | | | | |
| Incubation | HBSS | HBSS | HBSS | HBSS | HBSS |
| medium | supplemented | supplemented | supplemented | supplemented | supplemented |
| | with HEPES | with HEPES | with HEPES | with HEPES | with HEPES |
| | (9 mM) and | (9 mM) and | (9 mM) and | (9 mM) and | (9 mM) and |
| | sodium | sodium | sodium | sodium | sodium |
| | bicarbonate | bicarbonate | bicarbonate | bicarbonate | bicarbonate |
| | (4 mM), pH 7.4 | (4 mM), pH 7.4 | (4 mM), pH 7.4 | (4 mM), pH 7.4 | (4 mM), pH 8.5 |
| Number of | 3 | 3 | 3 | 3 | 3 |
| replicates | | | | | |
| Probe | LSC | LSC | LSC | LSC | LSC |
| substrate | | | | | |
| analysis | | | | | |
| method | | | | | |

The toxicity of Compound 1 to the various cell systems used in the study were assessed by measuring the lactate dehydrogenase (LDH) released from the cells into the medium. For Caco-2 and HEK293 cells, less than 25% cytotoxicity was observed. In MDCKII control cells, 100 and 600 μM Compound 1 were cytotoxic with percent cytotoxicity of 31.3 and 33.6%, respectively. As a result, 30 μM Compound 1 was the highest concentration analyzed for BCRP inhibition.

| | |
|---|---|
| BCRP | >30 |
| OATP1B1 | >600 |
| OATP1B3 | >600 |
| OAT1 | >600 |
| OAT3 | 174 |
| OCTI | >600 |
| MATE1 | >600 |
| MATE2-K | >600 |

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance:

Embodiment 1 provides a method of treating diabetic neuropathy or symptoms associated with diabetic neuropathy in an individual, the method comprising:

administering a therapeutically effective amount of a composition comprising a compound of Formula I:

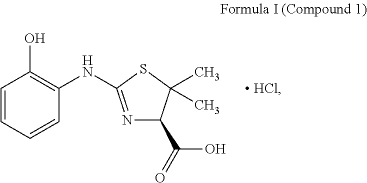 [SMILES: CC1(C)SC(Nc2ccccc2O)=N[C@@H]1C(=O)O]

to an individual having diabetic neuropathy or symptoms associated with diabetic neuropathy.

Embodiment 2 provides the method of embodiment 1, wherein the diabetic neuropathy comprises peripheral neuropathy, proximal neuropathy, autonomic neuropathy, focal neuropathy, or combinations thereof.

Embodiment 3 provides the method of any one of embodiments 1-2, wherein the individual has type I or type II diabetes.

Embodiment 4 provides the method of any one of embodiments 1-3, wherein the composition reduces or eliminates a symptom of peripheral neuropathy selected from the group consisting of numbness or insensitivity to pain or temperature, a tingling sensation, a burning sensation, a prickling sensation, sharp pains or cramps, extreme sensitivity to touch, and loss of balance and coordination.

Embodiment 5 provides the method of any one of embodiments 1-4, wherein the composition reduces or eliminates symptoms of muscle weakness, loss of reflexes, or postural changes associated with peripheral neuropathy.

Embodiment 6 provides the method of any one of embodiments 1-5, wherein the composition reduces or eliminates a symptom of proximal neuropathy selected from the group consisting of leg pain, hip pain, back pain, and radiculopathy (sciatica).

Embodiment 7 provides the method of any one of embodiments 1-6, wherein the composition reduces or eliminates symptoms of autonomic neuropathy selected from the group consisting of include hypoglycemia unawareness, blood pressure drops, increased heart rate, gastroparesis, constipation, diarrhea, urinary incontinence, decreased sexual response, poor temperature regulation, and light insensitivity.

Embodiment 8 provides the method of any one of embodiments 1-7, wherein the composition reduces or eliminates symptoms of neuropathy selected from the group consisting of pain in the lower back or pelvis, pain in the front of a thigh, pain in the chest, pain in the stomach, pain in the side of the body, pain on the outside of the shin or inside of the foot, pain in the chest, and pain in the abdomen.

Embodiment 9 provides the method of any one of embodiments 1-8, wherein the therapeutically effective amount comprises about 5 mg to about 5000 mg of Compound 1.

Embodiment 10 provides the method of any one of embodiments 1-9, wherein composition is administered for about 1 day to about 90 days.

Embodiment 11 provides the method of any one of embodiments 1-10, wherein administration of the composition results in a maximum observed plasma concentration (Cₘₐₓ) of about 5 μg/mL to about 300 μg/mL.

Embodiment 12 provides the method of any one of embodiments 1-11, wherein administration of the composition results in an area under the curve (AUC_{INF}) of about 100 hr·μg/mL to about 3000 hr·μg/mL.

Embodiment 13 provides the method of any one of embodiments 1-12, wherein the individual is human.

Embodiment 14 provides the method of any one of embodiments 1-13, wherein the composition comprises at least one additional pharmaceutically active agent.

Embodiment 15 provides the method of any one of embodiments 1-14, wherein the composition comprises at least one pharmaceutically acceptable excipient.

Embodiment 16 provides the method of any one of embodiments 1-15, wherein the composition comprises at least one pharmaceutically acceptable carrier.

Embodiment 17 provides the method of any one of embodiments 1-16, wherein the composition is administered to the individual by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous administration.

Embodiment 18 provides the method of any one of embodiments 1-17, wherein the composition is administered orally.

Embodiment 19 provides the method of any one of embodiments 1-18, wherein the composition is administered in a form comprising a tablet, hard capsule, soft capsule, cachet, troche, lozenge, or suppository.

Embodiment 20 provides a method of improving sleep quality in an individual having diabetic neuropathy or symptoms associated with diabetic neuropathy, the method comprising:

administering a therapeutically effective amount of a composition comprising a compound of Formula I:

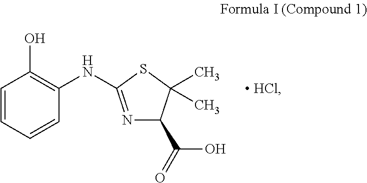 [SMILES: CC1(C)SC(Nc2ccccc2O)=N[C@@H]1C(=O)O]

to an individual having diabetic neuropathy or symptoms associated with diabetic neuropathy.

Embodiment 21 provides the method of embodiment 20, wherein the diabetic neuropathy comprises peripheral neuropathy, proximal neuropathy, autonomic neuropathy, focal neuropathy, or combinations thereof.

Embodiment 22 provides the method of any one of embodiments 20-21, wherein the individual has type I or type II diabetes.

Embodiment 23 provides the method of any one of embodiments 20-22, wherein the therapeutically effective amount comprises about 5 mg to about 5000 mg of Compound 1.

Embodiment 24 provides the method of any one of embodiments 20-23, wherein composition is administered for about 1 day to about 90 days.

Embodiment 25 provides the method of any one of embodiments 20-24, wherein administration of the composition results in a maximum observed plasma concentration (Cₘₐₓ) of about 1 μg/mL to about 300 μg/mL.

Embodiment 26 provides the method of any one of embodiments 20-25, wherein administration of the composition results in an area under the curve (AUC_{INF}) of about 10 hr·μg/mL to about 3000 hr·μg/mL.

Embodiment 27 provides the method of any one of embodiments 20-26, wherein the individual is human.

1. A method of ameliorating diabetic neuropathy or symptoms associated with diabetic neuropathy in an individual, the method comprising:
administering a therapeutically effective amount of a composition comprising a compound of Formula I: 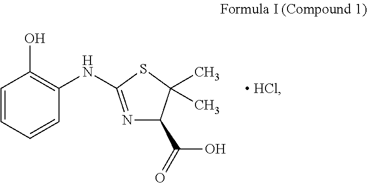 [SMILES: CC1(C)SC(Nc2ccccc2O)=N[C@@H]1C(=O)O]
to an individual having diabetic neuropathy or symptoms associated with diabetic neuropathy.

2. The method of claim 1, wherein the diabetic neuropathy comprises peripheral neuropathy, proximal neuropathy, autonomic neuropathy, focal neuropathy, or combinations thereof.

3. The method of claim 1, wherein the individual has type I or type II diabetes.

4. The method of claim 2, wherein the composition reduces or eliminates a symptom of peripheral neuropathy selected from the group consisting of numbness or insensitivity to pain or temperature, a tingling sensation, a burning sensation, a prickling sensation, sharp pains or cramps, extreme sensitivity to touch, and loss of balance and coordination.

5. The method of claim 2, wherein the composition reduces or eliminates symptoms of muscle weakness, loss of reflexes, or postural changes associated with peripheral neuropathy.

6. The method of claim 2, wherein the composition reduces or eliminates a symptom of proximal neuropathy selected from the group consisting of leg pain, hip pain, back pain, and radiculopathy (sciatica).

7. The method of claim 2, wherein the composition reduces or eliminates symptoms of autonomic neuropathy selected from the group consisting of include hypoglycemia unawareness, blood pressure drops, increased heart rate, gastroparesis, constipation, diarrhea, urinary incontinence, decreased sexual response, poor temperature regulation, and light insensitivity.

8. The method of claim 2, wherein the composition reduces or eliminates symptoms of neuropathy selected from the group consisting of pain in the lower back or pelvis, pain in the front of a thigh, pain in the chest, pain in the stomach, pain in the side of the body, pain on the outside of the shin or inside of the foot, pain in the chest, and pain in the abdomen.

9. The method of claim 1, wherein the therapeutically effective amount comprises about 5 mg to about 5000 mg of Compound 1.

10. The method of claim 1, wherein composition is administered for about 1 day to about 90 days.

11. The method of claim 1, wherein administration of the composition results in a maximum observed plasma concentration (Cₘₐₓ) of about 5 μg/mL to about 300 μg/mL.

12. The method of claim 1, wherein administration of the composition results in an area under the curve (AUC_{INF}) of about 100 hr·μg/mL to about 3000 hr·μg/mL.

13. The method of claim 1, wherein the individual is human.

14. The method of claim 1, wherein the composition comprises at least one additional pharmaceutically active agent.

15. The method of claim 1, wherein the composition comprises at least one pharmaceutically acceptable excipient.

16. The method of claim 1, wherein the composition comprises at least one pharmaceutically acceptable carrier.

17. The method of claim 1, wherein the composition is administered to the individual by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous administration.

18. The method of claim 17, wherein the composition is administered orally.

19. The method of claim 18, wherein the composition is administered in a form comprising a tablet, hard capsule, soft capsule, cachet, troche, lozenge, or suppository.

20. A method of improving sleep quality in an individual having diabetic neuropathy or symptoms associated with diabetic neuropathy, the method comprising:
administering a therapeutically effective amount of a composition comprising a compound of Formula I: 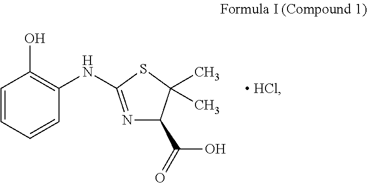 [SMILES: CC1(C)SC(Nc2ccccc2O)=N[C@@H]1C(=O)O]
to an individual having diabetic neuropathy or symptoms associated with diabetic neuropathy.

21. The method of claim 20, wherein the diabetic neuropathy comprises peripheral neuropathy, proximal neuropathy, autonomic neuropathy, focal neuropathy, or combinations thereof.

22. The method of claim 20, wherein the individual has type I or type II diabetes.

23. The method of claim 20, wherein the therapeutically effective amount comprises about 5 mg to about 5000 mg of Compound 1.

24. The method of claim 20, wherein composition is administered for about 1 day to about 90 days.

25. The method of claim 20, wherein administration of the composition results in a maximum observed plasma concentration (Cₘₐₓ) of about 1 μg/mL to about 300 μg/mL.

26. The method of claim 20, wherein administration of the composition results in an area under the curve (AUC_{INF}) of about 10 hr·μg/mL to about 3000 hr·μg/mL.

27. The method of claim 20, wherein the individual is human.
